SEQUENCE LISTING (28 sequences)

SEQ ID NO: 1 - protein, 195 aa, Homo sapiens
  Human BID
MDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 2 - protein, 197 aa, Artificial Sequence
  Human BID engineered with HCV protease cleavage sequence.
MDCEVNNGSSLRDECITNLLVFGFLQSCSDNSFRRELDALGHELPVLAPQWEGYDELQTEGAEDVVCCSMSYSIEAESESQEDIIRNIARHLAQVGDSMERSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 3 - protein, 199 aa, Artificial Sequence
  Human BID engineered with HIV-A protease cleavage sequence.
MDCEVNNGSSLRDECITNLLVFGFLQSCSDNSFRRELDALGHELPVLAPQWEGYDELQTEGSQVSQNYPIVQNLQIEAESESQEDIIRNIARHLAQVGDSMERSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 4 - protein, 277 aa, Homos sapiens
  Human procaspase-3
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 5 - protein, 284 aa, Artificial Sequence
  Human procaspase-3 engineered with HCV protease cleavage sequence.
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGAEDVVCCSMSYSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 6 - protein, 496 aa, Homo sapiens
  Human procasepase-8
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRFHFCRMSWAEANSQCQTQSVPFWRRVDHLLIRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 7 - protein, 544 aa, Artificial Sequence
  Human procaspase-8 engineered with HCV protease cleavage sequence.
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQEFSKGEELCGVMAEDVVCCSMSYSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPAEDVVCCSMSYSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 8 - protein, 10 aa, Hepatitis C virus
  Hepatitis C protease site
DLEVVTSTWV

SEQ ID NO: 9 - protein, 10 aa, Hepatitis C virus
  HCV protease site
DEMEECASHL

SEQ ID NO: 10 - protein, 10 aa, Hepatitis C virus
  HCV protease site
DCSTPCSGSW

SEQ ID NO: 11 - protein, 10 aa, Hepatitis C virus
  HCV protease site
EDVVCCSMSY

SEQ ID NO: 12 - protein, 14 aa, Human immunodeficiency virus-1
  HIV-1 protease site
SQVSQNYPIVQNLQ

SEQ ID NO: 13 - protein, 14 aa, Human immunodeficiency virus I
  HIV-1 protease cleavage site
CTERQANFLGKIWP

SEQ ID NO: 14 - protein, 14 aa, Human immunodeficiency virus-1
  HIV-1 protease cleavage site
GTVSFSFPQITLWQ

SEQ ID NO: 15 - protein, 14 aa, Human immunodeficiency virus
  HIV-1 protease cleavage site
IGCTLNFPISPIET

SEQ ID NO: 16 - protein, 20 aa, Hepatitis C virus
  HCV protease cleavage site
CMSADLWVVTSTWVLVGGVL

SEQ ID NO: 17 - protein, 20 aa, Hepatitis C virus
  HCV protease cleavage site
YQEFDEMEECASHLPYIEQG

SEQ ID NO: 18 - protein, 20 aa, Hepatitis C virus
  HCV protease cleavage site
WISSECTTPCSGSWLRDIWD

SEQ ID NO: 19 - protein, 20 aa, Hepatitis C virus
  HCV protease cleavage site
GADTEDVVCCSMSYTWTGAL

SEQ ID NO: 20 - protein, 12 aa, Hepatitis C virus
  HCV protease cleavage site
AEDVVCCSMSYS

SEQ ID NO: 21 - protein, 14 aa, Human immunodeficiency virus
  HIV-A protease cleavage site
SQVSQNYPIVQNLQ

SEQ ID NO: 22 - protein, 14 aa, Human immunodeficiency virus
  HIV-D protease cleavage site
CTERQANFLGKIWP

SEQ ID NO: 23 - protein, 14 aa, Hepatitis C Virus
  HCV protease cleavage site
SQVSQNYPIVQNLQ

SEQ ID NO: 24 - protein, 14 aa, Hepatitis C virus
  HCV protease site
CTERQANFLGKIWP

SEQ ID NO: 25 - protein, 14 aa, Hepatitis C virus
  HCV protease site
IGCTLNFPISPIET

SEQ ID NO: 26 - DNA, 588 bp, Homo sapiens
  CDS (1)...(588): Human BID coding sequence
atggactgtgaggtcaacaacggttccagcctcagggatgagtgcatcacaaacctactggtgtttggcttcctccaaagctgttctgacaacagcttccgcagagagctggacgcactgggccacgagctgccagtgctggctccccagtgggagggctacgatgagctgcagactgatggcaaccgcagcagccactcccgcttgggaagaatagaggcagattctgaaagtcaagaagacatcatccggaatattgccaggcacctcgcccaggtcggggacagcatggaccgtagcatccctccgggcctggtgaacggcctggccctgcagctcaggaacaccagccggtcggaggaggaccggaacagggacctggccactgccctggagcagctgctgcaggcctaccctagagacatggagaaggagaagaccatgctggtgctggccctgctgctggccaagaaggtggccagtcacacgccgtccttgctccgtgatgtctttcacacaacagtgaattttattaaccagaacctacgcacctacgtgaggagcttagccagaaatgggatggactga

SEQ ID NO: 27 - protein, 199 aa, Artificial Sequence
  Human proBID engineered with HIV-D protease cleavage sequence.
MDCEVNNGSSLRDECITNLLVFGFLQSCSDNSFRRELDALGHELPVLAPQWEGYDELQTEGCTERQANFLGKIWPIEAESESQEDIIRNIARHLAQVGDSMERSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 28 - DNA, 834 bp, Homo sapiens
  CDS (1)...(834): Human procaspase3 coding sequence
atggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaa

(19) United States
(12) Patent Application Publication
(10) Pub. No.: US 2004/0043949 A1
(43) Pub. Date: Mar. 4, 2004

(54) Therapeutic system targeting pathogen proteases and uses thereof

(76) Inventors: Christopher Richardson, Toronto (CA); Eric C. Hsu, Vancouver (CA); David Lorne Tyrrell, Edmonton (CA); Norman M. Kneteman, Edmonton (CA)

Correspondence Address: BOZICEVIC, FIELD & FRANCIS LLP, 200 MIDDLEFIELD RD, SUITE 200, MENLO PARK, CA, 94025, US

(21) Appl. No.: 10/232,884

(22) Filed: Aug. 30, 2002

(51) Int. Cl. A61K 48/00; C07H 21/04; C07H 21/02

(52) U.S. Cl. 514/44; 536/23.2; 536/23.72

(57) ABSTRACT

The invention provides anti-pathogen polypeptide and polynucleotide compositions, and methods of use. In general, the composition of the invention provide a modified pro-polypeptide comprising a pro-domain, a pathogen protease cleavage site, and a cytotoxic domain which can be activated by cleavage of the pro-polypeptide by a protease of an intracellular pathogen. The invention further provides nucleic acids encoding the subject polypeptides, and vectors and host cells comprising the subject nucleic acids. Cleavage of the pro-polypeptide by the pathogen protease results in activation of the cytotoxic domain, and decreases the viability of the pathogen-infected host cell. Methods for using the subject nucleic acids and polypeptides to reduce the viability of a pathogen-infected cell, and for reducing the pathogen load of a subject infected with a pathogen are provided. The invention further provides kits for carrying out the subject methods.

### FIELD OF THE INVENTION

The field of the invention relates to anti-pathogen compositions, particularly those that are activated in infected host cells.

### BACKGROUND OF THE INVENTION

Modern medicine has struggled to find effective treatments for pathogens that have at least part of their replication cycle inside host cells. Such intracellular pathogens present particular challenges, since effective therapy must result not only in elimination of extracellular infectious agents in the patient's blood stream and other bodily fluids, but must also effectively rid of pathogens that reside within host cells. This latter aspect of therapy for treatment of an intracellular pathogen presents particular challenges, since drugs that act on targets that located inside host cells are often toxic to both infected and uninfected host cells. Futhermore, intracellular pathogens often establish persistent and latent infections within a cell in order to a host's immune system.

Viral pathogens, such as hepatitis C virus (HCV) and human immunodeficiency virus (HIV), are examples of intracellular pathogens that can be difficult to treat. HCV is the principal etiological agent of post-transfusion and community-acquired non-A non-B hepatitis worldwide. It is estimated that over 150 million people worldwide are infected by the virus. A high percentage of carriers become chronically infected with this pathogen and many patients progress to a state of chronic liver disease, so-called chronic hepatitis C. This group is in turn at high risk for serious liver disease such as liver cirrhosis, hepatocellular carcinoma and terminal liver disease leading to death.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins. In the case of HCV, the generation of mature nonstructural proteins (NS2, NS3, NS4A, NS4B, NS5A, and NS5B) is effected by two viral proteases. The first one is a metalloprotease located in NS2 that cleaves the NS2-NS3 junction in cis; the second one is a serine protease contained within the N-terminal region of NS3 (henceforth referred to as NS3 protease) and mediates all the subsequent cleavages downstream of NS3, both in cis, at the NS3-NS4A cleavage site, and in trans, for the remaining NS4A-NS4B, NS4B-NS5A, NS5A-NS5B sites. The NS4A protein appears to serve multiple functions, acting as a cofactor for the NS3 protease and possibly assisting in the membrane localization of NS3 and other viral replicase components.

The mechanism by which HCV establishes viral persistence and causes a high rate of chronic liver disease has not been elucidated. It is not known how HCV interacts with and evades the host immune system. In addition, the roles of cellular and humoral immune responses in protection against HCV infection and disease have yet to be established. Immunoglobulins have been reported for prophylaxis of transfusion-associated viral hepatitis, however, the Center for Disease Control does not presently recommend immunoglobulins treatment for this purpose. The lack of an effective protective immune response is hampering the development of a vaccine or adequate post-exposure prophylaxis measures, so in the near-term, hopes are firmly pinned on antiviral interventions. Such antiviral interventions to date have focused upon, for example, ribavirin and interferon-alpha (IFN-α)-based therapy, either alone or in combination with ribavirin. However, not all patients are responsive to these therapies, and new drugs for treatment of chronic HCV are of great interest in the field.

More recently several strategies have been developed for specifically inhibiting the HCV encoded protease and replication enzymes, causing a reduction in the rate of reproduction of the virus. However, the genome of HCV (and most other RNA viruses such as HIV) exhibits significant genetic heterogeneity as a result of the accumulation of mutations during viral replication. This high mutation rate can be attributed to an error-prone RNA-dependent RNA polymerase that lacks proofreading activity and, as such, HCV circulates in an infected individual as a population of closely related, yet heterogeneous, sequences: the quasispecies. The distribution of mutations has been reported to be uneven but the basis of this variability is unexplained. The protease activities between most HCV strains and genotypes are well conserved, making the protease a viable target for antiviral therapy by small molecule inhibitors. In fact, the proteases, RNA helicases, and polymerases are very similar between different HCV isolates and have been touted as valid antiviral targets (Randall et al., (2002) Curr. Opin. Infect. Dis. 14: 743-747; Di Bisceglie et al., (2002) Hepatology 35: 224-231). However, minor changes in the polymerase or protease can provide resistance against current antiviral agents such as AZT, ddl, 3TC, and indinavir (Condra, J. H. (1998) Haemophilia 4: 610-615; Brenner et al., (2002) Ann. N.Y. Acad. Sci. 918: 9-15). As such, a multidrug or “cocktail” approach has been adopted to overcome drug resistance. Similar strategies for combating other intracellular pathogens such as HIV have also faced difficulties, particularly because the HIV genome has a high mutation rate.

As such, a great need exists for new methods for combating intracellular infections, particularly HCV and HIV infections. The described invention meets these, and other, needs.

### LITERATURE

References of interest include U.S. Pat. Nos. 6,221,355, 5,554,528, and publications: Baltimore, Nature: 335:739-745 (1988), Harrison et al, Human Gene Therapy 3:461, (1992), Clarke et al., J. Gen. Virol. 78, 2397-2410 (1997), Major et al., Hepatology 25, 1527-1538 (1997), Purcell et al., Hepatology 26, 11s-14s (1997), Bartenschlager, Intervirology 40, 378-393 (1997), Blight et al., Antivir Ther 3, 71-81 (1998), Rosen et al., Mol Med Today 5, 393-399 (1999), Nature 396, 119-122 (1998), Li et al., Cell 94, 491-501 (1998), Han et al., J Biol Chem 272, 13432-13436 (1997), Luo et al., Cell 94, 481-490 (1998), J Gen Virol 81 Pt 11, 2573-2604 (2000), Hitt et al., Adv Virus Res 55, 479-505 (2000), Connelly, Curr Opin Mol Ther 1, 565-572 (1999), Wilson, Adv Drug Deliv Rev 46, 205-209 (2001) and Mercer, Nat Med 7, 927-933 (2001).

### SUMMARY OF THE INVENTION

The invention provides anti-pathogen polypeptide and polynucleotide compositions, and methods of use. In general, the composition of the invention provide a modified pro-polypeptide comprising a pro-domain, a pathogen protease cleavage site, and a cytotoxic domain which can be activated by cleavage of the pro-polypeptide by a protease of an intracellular pathogen. The invention further provides nucleic acids encoding the subject polypeptides, and vectors and host cells comprising the subject nucleic acids. Cleavage of the pro-polypeptide by the pathogen protease results in activation of the cytotoxic domain, and decreases the viability of the pathogen-infected host cell. Methods for using the subject nucleic acids and polypeptides to reduce the viability of a pathogen-infected cell, and for reducing the pathogen load of a subject infected with a pathogen are provided. The invention further provides kits for carrying out the subject methods.

The invention takes advantage of the fact that many intracellular pathogens produce proteases that are specific for polypeptides of the pathogen, and do not act on host cell proteins. For example, many viruses, e.g. picomaviruses, togaviruses, and flaviviruses, encode and synthesize their encoded proteins as a polyprotein, which is cleaved by specific viral proteases with specificity for sequences within the polyprotein.

A feature of the invention is that the anti-pathogen nucleic acid compositions specifically reduce the viability of cells having an intracellular pathogen-encoded protease, and, as such, the compositions and methods may be used to effectively treat pathogen-infected subjects.

Another feature of the subject invention is that the compositions of the invention are particularly effective against viral pathogens, such as RNA viruses, and are effective against various genotypes and quasispecies of RNA viruses due to the highly conserved nature of the viral-specific protease and its activity.

One advantage of the invention, is that the methods and compositions provide for reduction in viability of pathogen-infected cells, without substantially affecting viability of uninfected host cells.

Another advantage is, since the protease cleavage site and proteases of intracellular pathogens can be expected to be well-conserved across different serotypes and genotypes, the methods and compositions of the invention can be effective against these serotypes and genotypes.

Still another advantage is that a protease cleavage site that is native to the infecting pathogen is the substrate for the protease and activates the “drug” of the cytotoxic domain. Thus, resistant pathogen strains must have at least two complementary mutations—in both the protease cleavage site and in the protease itself, in order to overcome the effects of the engineered pro-peptide drug. This greatly reduces the probability of the development of resistant strains which require an active protease for their maturation.

These and other advantages and features of the invention will become apparent to those persons skilled in the art upon reading the details of the anti-pathogen compositions as more fully described below.

### BRIEF DESCRIPTION OF THE DRAWINGS

FIG. 1 schematically shows procaspase 3, procaspase 8 and BID molecules that have been engineered to contain specific cleavage sites recognized by the HSV NS3 serine protease.

FIG. 2 is a series of photographs (panels A-E) showing H9C2 rat muscle fibroblast cells transfected recombinant retroviruses and adenoviruses of the invention. Cells were examined using Hofmann polarized light microscopy (left panels) or UV fluorescence microscopy (right panels). FIG. 2, panel A: Cells were infected with retrovirus (NS4A-NS3), and subsequently with control recombinant adenovirus that did not contain a foreign gene. FIG. 2, panel B: Cells were infected with control retrovirus expressing EGFP, and subsequently with adenovirus expressing modified caspase3. FIG. 2, panel C: Cells were infected with retrovirus expressing NS4A-NS3, followed by adenovirus expressing modified caspase 3. FIG. 2, panel D: Cells were infected with control retrovirus expressing EGFP, followed by adenovirus expressing modified BID. FIG. 2, panel E: Cells were infected with retrovirus expressing NS4A-NS3, followed by infection with adenovirus expressing modified BID.

FIG. 3 is a photograph (panel A), a line graph (panel B), and bar graphs (panels C and D) showing results of Example 3. FIG. 3, panel A: Cell lysates from H9C2 cells expressing both NS3 protease and modified BID containing the FLAG epitope at its carboxyl terminus were subjected to immunoprecipitation with anti-FLAG-agarose beads. Immunoblot analysis was subsequently performed using monoclonal antibody against human BID: lane 1, cells infected with retrovirus-NS3 and susbsequently with adenovirus control that did not express a foreign gene; lane 2, cells infected with retrovirus-NS3 and subsequently with adenovirus containing modified BID; lane 3, cells infected with retrovirus-EGFP and subsequently with the adenovirus control; lane 4, cells infected with retrovirus-EGFP and subsequently with adenovirus-modified BID. Modified BID was cleaved in the presence of NS3 protease (lane 2). FIG. 3, panel B: Caspase 3 activity of H9C2 cells expressing either the NS3 serine protease, modified BID. Cells containing both modified BID and NS3 protease exhibited increased caspase 3 activity. FIG. 3, panel C: H9C2 cells expressing either the serine protease, modified BID or both were stained with Annexin V, a marker for apoptosis, at 24 hours post infection. The percentage of Annexin V positive cells was determined using FACS analysis. When both NS3 protease and modified BID were present, over 50% of the cells were positive for Annexin V. FIG. 3, panel D: H9C2 cells expressing either the NS3 serine protease, modified BID, or both, in the presence of different amounts of VAD-fmk (caspase inhibitor) were stained with Annexin V at 24 hours post infection. The percentage of Annexin V positive cells was determined using FACS analysis. The VAD-fmk inhibitor decreased the amount of apoptosis as measured through Annexin V staining.

FIG. 4 is a series of photographs (panels A-L). Huh7 human hepatocytes transfected with HCV genome or Huh7 cell lines containing HCV replicons underwent apoptosis when infected with modified BID-adenovirus. Huh7 cells were transfected with HCV genome genotype 1 a, followed by infection with adenovirus control that did not express a foreign gene, or recombinant adenovirus that expressed modified BID molecules. 36 hours after adenovirus infection, morphological changes of the Huh7 cells were observed by fluorescence microscopy. FIG. 4, panel A: Transfection of cells with control vector only, followed by infection with recombinant adenovirus expressing modified BID. FIG. 4, panel B: Cells transfected with HCV genome, followed by wild-type adenovirus infection. FIG. 4, panel C:Cells transfected with HCV genome, followed by infection with recombinant adenovirus expressing modified BID. Arrows indicate dead cells. FIG. 4, panel D: Cells transfected with gene for NS3 protease, followed by infection with recombinant adenovirus expressing modified BID. Arrows indicate dead cells. HCV replicon cell lines were infected with either control adenovirus or recombinant adenovirus expressing modified BID. Morphological changes of the cells were observed 24 hours post adenovirus infection using phase contrast microscopy. FIG. 4, panel E: Huh7 cells containing the HBI-10A replicon were infected with control adenovirus. FIG. 4, panel F: Huh7 cells containing the HBI-10A replicon were infected with adenovirus expressing modified BID. FIG. 4, panel G: Huh7 cells containing the HBIII-27 replicon were infected with control adenovirus. FIG. 4, panel H: Huh7 cells containing the HBIII-27 replicon were infected with adenovirus expressing modified BID. FIG. 4, panels I and J: Huh7 cells infected with control adenovirus or recombinant virus expressing modified BID, respectively. FIG. 4, panel K: Immunoblot analysis with anti-HCV core antibody indicates that viral proteins are expressed and processed in the Huh7 cells transfected with the HCV genome. Lane 1, cells transfected with pcDNA 1.1-HCV core expression vector; lane 2, cells transfected with pcDNA1.1 vector alone; lane 3, cells transfected with HCV genome genotype la. FIG. 4, panel L: The replicon cell lines and their parental Huh7 cells were lysed, and immunoblot analysis was performed using monoclonal antibody against HCV NS3 protease. Lane 1, HBI-10A replicon cell line; lane 2, HBIII-27 replicon cell line; and lane 3, parental Huh7 cells.

FIG. 5 is a series of photographs, panels A-F. Modified BID molecules may be used prophylacticly to protect hepatocytes from challenge at low multiplicity of infection with a hepatitis C virus model. Huh7 cells were infected with either control adenovirus or recombinant adenovirus expressing modified BID for 24 hours. These cells were subsequently challenged with either wild type Sindbis virus or chimeric Sindbis virus (MutA) at a MOI of 0.1 pfu/cell. After 72 hours post-infection with Sindbis virus, changes in cell morphology were evaluated by phase contrast microscopy. FIG. 5, panel A: Huh7 cells were infected with control recombinant adenovirus that did not express a foreign gene. No cell death was apparent. FIG. 5, panel B: Huh7 cells were infected-with a recombinant adenovirus expressing modified BID. No cell death was apparent. FIG. 5, panel C: Cells infected with control adenovirus that did not express a foreign gene and subsequently with the chimeric Sindbis virus (Mut A). Cell death was apparent. FIG. 5, panel D: Cells were initially infected with adenovirus expressing modified BID and subsequently challenged with chimeric Sindbis virus (Mut A). No cell death was apparent. FIG. 5, panels E and F: Cells were infected with control adenovirus that did not express a foreign gene or recombinant adenovirus expressing modified BID, respectively, and were subsequently challenged with wild type Sindbis virus. Cell death was apparent in both cases.

FIG. 6 is a series of series of schematics, panels A-C, showing sequence alignments of FIG. 6, panel A: BID (top; SEQ ID NO:1) and modified BID with an HCV NS3 cleavage site (bottom; SEQ ID NO:2), FIG. 6, panel B: procaspase 3 (top; SEQ ID NO:4) and modified procaspase 3 with a HCV NS3 cleavage site (bottom; SEQ ID NO:5) and FIG. 6, panel C: BID (top; SEQ ID NO:1) and modified BID with an HIV protease cleavage site (bottom; SEQ ID NO:3 and SEQ ID NO:27).

FIG. 7 shows fluorescent images of liver cells of SCID mice injected with adenovirus GFP.

FIG. 8 shows a timeline for the administration of a vector encoding modified BID and control vectors and analysis performed.

FIG. 9 shows a graph of changes in human-alpha 1 anti-trypsin after adenovirus-mBID administration.

FIG. 10 shows a graph of changes in HCV viral load after adenovirus-mBID administration.

FIG. 11 shows a graph of changes in HCV viral load after adenovirus-mBID administration for individual mice.

### DEFINITIONS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a protease” includes a plurality of such proteases and reference to “the protease cleavage site” includes reference to one or more protease cleavage sites and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

By the term “pro-polypeptide” as used herein is meant any naturally occurring polypeptide or variant thereof that can be cleaved at a protease cleavage site by a sequence-specific protease. In general, a pro-polypeptide has a pro-domain and a domain with biological activity (e.g., a cytotoxic activity), separated by a protease cleavage site. Cleavage of the pro-polypeptide results in two products: a released pro-domain and a mature polypeptide. Release of the mature polypeptide by cleavage provides for “activation” of a cytotoxic activity of the mature polypeptide, e.g., cleavage allows for accessibility of one or more active sites on the mature polypeptide for substrate binding. The pro-polypeptide generally has not detectable or relatively low cytotoxic activity relative to the mature polypeptide. As such, a pro-polypeptide is a type of “cleavage-dependent cytotoxic polypeptide”. Examples of pro-polypeptides of particular interest include are zymogens, particularly proteolytic zymogens, toxins that are activated by proteolysis, and pro-apoptotic molecules that are activated by proteolysis such as BID, procaspase 3 and procaspase 8.

A “pro-polypeptide element” refers to a pro-domain, protease cleavage site, or mature polypeptide (e.g., cleavage-dependent cytotoxic polypeptide).

By “cleavage-dependent cytotoxic polypeptide” is meant a polypeptide that, upon release from a pro-polypeptide or modified pro-polypeptide of the invention by protease cleavage, exhibits or mediates a cytotoxic effect upon a host cell in which the cleavage-dependent cytotoxic polypeptide is present. The “cleavage-dependent cytotoxic polypeptide” is often referred to herein as the “cytotoxic domain” of a pro-polypeptide or modified pro-polypeptide.

“Cytotoxic effects” or “cytotoxic activity” refers to effects or activities that facilitate reduction of host cell viability, including cell death. Such effects and activities may be associated with, for example, induction of apoptosis in the host cell, reduction of host cell protein synthesis, reduction in host cell transcription, genomic DNA fragmentation, membrane disintegration, breakdown of the nuclear lamina, change in potential of a cell and the like.

By “modified pro-polypeptide”, also referred to herein as a “pathogen protease cleavage-dependent cytotoxic polypeptide”, is meant a pro-polypeptide that has been modified to have a non-native protease cleavage site and, in some embodiments having one or more inactivated endogenous protease cleavage sites (i.e., the protease cleavage site is “inactivated” in that it is not cleaved or cleaved at detectable levels by the cellular protease that cleaves the unmodified endogenous protease cleavage site). Usually, the amino acid sequence of a pro-polypeptide is altered, particularly with respect to a protease cleavage site. In general, a modified pro-polypeptide of the invention has a protease cleavage site that is not native to the pro-polypeptide and positioned within the modified pro-polypeptide such that action of a sequence-specific protease upon the cleavage site results in release of the pro-domain and mature polypeptide, the latter of which exhibits a biological activity that is cytotoxic to a mammalian host cell. In most cases, the pro-domain and cytotoxic domain of a modified pro-polypeptide are native to a pro-polypeptide

A “variant” of a pro-polypeptide or pro-polypeptide element (e.g., pro-domain, mature polypeptide) is defined as a pro-polypeptide that is altered by one or more amino acid residues. Such variants can have “conservative” changes relative to a reference pro-polypeptide or pro-polypeptide element amino acid sequence, wherein a substituted amino acid has similar structural or chemical properties, e.g., replacement of leucine with isoleucine. In some embodiments, such variants can have “nonconservative” changes, e.g., replacement of a glycine with a tryptophan. Similar variations can also include amino acid deletions or insertions, or both. Variants of a pro-polypeptide or pro-polypeptide element retain the relevant basic structural features (e.g., a variant pro-polypeptide a pro-domain, a cleavage site, and a cleavage activatable cytotoxic domain (mature polypeptide)) and biolgoical activity (e.g., cytotoxic activity of the cleavage-activatable cytotoxic domain) of a pro-polypeptide.

Guidance in determining which and how many amino acid residues may be substituted, inserted or deleted (e.g., without abolishing cytotoxic activity of a cleavage-activatable cytotoxic domain) may be found by comparing the sequence of the pro-polypeptide or pro-polypeptide element to known pro-polypeptides with a related structure and function (e.g. a mouse BID homolog of GenBank accession number NP031570, a chicken BID homolog of GenBank accession number AAM48284, or a rat BID homolog of GenBank accession number NP_{—}073175 may be used as guidance for modifying human BID). Assays for cytotoxicity are readily available and straightforward, and can be readily applied to determine which and how many amino acid residues may be substituted, inserted or deleted may be determined empirically.

A “deletion” is defined as a change in either amino acid or nucleotide sequence in which one or more amino acid or nucleotide residues, respectively, are absent as compared to an amino acid sequence or nucleotide sequence of a naturally occurring pro-polypeptide. In the context of a pro-polypeptide and pro-polypeptide element amino acid or polynucleotide sequence, a deletion can involve deletion of about 2, about 5, about 10, up to about 20, up to about 30 or up to about 50 or more amino acids. A pro-polypeptide may contain more than one deletion.

An “insertion” or “addition” is that change in an amino acid or nucleotide sequence which has resulted in the addition of one or more amino acid or nucleotide residues, respectively, as compared to an amino acid sequence or nucleotide sequence of a naturally occurring pro-polypeptide. “Insertion” generally refers to addition to one or more amino acid residues within an amino acid sequence of a polypeptide, while “addition” can be an insertion or refer to amino acid residues added at the N- or C-termini. In the context of a pro-polypeptide and pro-polypeptide element amino acid or polynucleotide sequence is an insertion or addition of up to about 10, up to about 20, up to about 30 or up to about 50 or more amino acids. A pro-polypeptide may contain more than one insertion.

A “substitution” results from the replacement of one or more amino acids or nucleotides by different amino acids or nucleotides, respectively as compared to an amino acid sequence or nucleotide sequence of a naturally occurring pro-polypeptide. It is understood that the pro-polypeptide or pro-polypeptide element may have conservative amino acid substitutions which have substantially no effect on cytotoxic activity. By conservative substitutions is intended combinations such as gly, ala; val, ile, leu; asp, glu; asn, gln; ser, thr; lys, arg; and phe, tyr.

The term “biologically active” pro-polypeptide or pro-polypeptide element refers to a pro-polypeptide having structural and biochemical functions of a naturally occurring pro-polypeptide or pro-polypeptide element.

“Non-native”, “non-endogenous”, and “heterologous” in the context of protease cleavage sites in the modified pro-polypeptides of the invention are used interchangeably herein to refer to a protease cleavage site derived from a polypeptide other than the pro-polypeptide selected for modification.

Unless specifically indicated otherwise, the term “protease” in the context of the anti-pathogen systems of the invention is meant a sequence specific protease that recognizes and cleaves a specific motif in the sequence of a pro-polypeptide or modified pro-polypeptide. A protease may be a cellular protease, i.e. a protease encoded by a genome endogenous to a cell, e.g., a granzyme, or a pathogen protease i.e. a protease encoded by a genome of a pathogen, e.g., the HIV or HCV protease.

By “intracellular pathogen” is a pathogen that has at least part of its replication cycle within a cell of an infected host. In most embodiments an intracellular pathogen is an obligate intracellular pathogen, meaning that the pathogen must reside within a host cell to facilitate pathogen replication. Examples of intracellular pathogens are viruses, including HIV and HCV.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially pure” refers to a compound that is removed from its natural environment and is at least 60% free, preferably 75% free, and most preferably 90% free from other components with which it is naturally associated.

A “coding sequence” or a sequence that “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence.

“Operably linked” refers to an arrangement of elements wherein the components so described are configured so as to perform their usual function. Thus, a given pro-polypeptide that is operably linked to a protease site is capable of being cleaved by a protease that recognizes the protease site so as to release a mature polypeptide having a biological activity of interest (e.g., cytotoxicity). In the case of a promoter, a promoter that is operably linked to a coding sequence will effect the expression of a coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” comprises any nucleic acid construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

Techniques for determining nucleic acid and amino acid “sequence identity” are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary-implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 85%-90%, more preferably at least about 90%-95%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., infra; DNA Cloning, supra; Nucleic Acid Hybridization, supra:

Two nucleic acid fragments are considered to “selectively hybridize” as described herein. The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization. A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning. A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH 7.6), 5× Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

A first polynucleotide is “derived from” a second polynucleotide if it has the same or substantially the same nucleotide sequence as a region of the second polynucleotide, its CDNA, complements thereof, or if it displays sequence identity as described above.

A first polypeptide is “derived from” a second polypeptide if it is (i) encoded by a first polynucleotide derived from a second polynucleotide, or (ii) displays sequence identity to the second polypeptides as described above. The term “unit dosage form” as used herein refers to physically discrete units suitable as unitary dosages for subjects (e.g., animals, usually humans), each unit containing a predetermined quantity of an agent, e.g. a plasmid in an amount sufficient to produce the desired effect in association with a pharmaceutically acceptable diluent, carrier or vehicle. The specifications for the novel unit dosage forms of the present invention will depend on a variety of factors including, but not necessarily limited to, the particular agent employed and the effect to be achieved, and the pharmacodynamics associated with each compound in the host.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of modified pro-polypeptide-enciding nucleic acids that can provide for enhanced or desirable effects in the subject (e.g., reduction of pathogen load, increase in CD4 count, reduction of disease symptoms, etc.).

“Subject”, “host” and “patient” are used interchangeably herein, to refer to an animal, human or non-human, susceptible to or having an infection by an intracellular pathogen and amenable to therapy according to the methods of the invention. Generally, the subject is a mammalian subject. Exemplary subjects include, but are not necessarily limited to, humans, cattle, sheep, goats, pigs, dogs, cats, and horses, with humans being of particular interest.

### DETAILED DESCRIPTION OF THE INVENTION

The invention provides anti-pathogen polypeptide and polynucleotide compositions, and methods of use. In general, the composition of the invention provide a modified pro-polypeptide comprising a pro-domain, a pathogen protease cleavage site, and a cytotoxic domain which can be activated by cleavage of the pro-polypeptide by a protease of an intracellular pathogen. The invention further provides nucleic acids encoding the subject polypeptides, and vectors and host cells comprising the subject nucleic acids. Cleavage of the pro-polypeptide by the pathogen protease results in activation of the cytotoxic domain, and decreases the viability of the pathogen-infected host cell. Methods for using the subject nucleic acids and polypeptides to reduce the viability of a pathogen-infected cell, and for reducing the pathogen load of a subject infected with a pathogen are provided. The invention further provides kits for carrying out the subject methods.

The present invention is based upon the observation that polynucleotide sequences encoding several protease cleavage-dependent cytotoxic molecules may be used to reduce the viability of cells infected with a pathogen. In most embodiments the molecules encode a pro-polypeptide that has been engineered to contain a protease cleavage site that is specific for the pathogen, and upon cleavage of the pro-polypeptide by the pathogen protease, a cytotoxic domain of the pro-polypeptide is activated and the cell containing the pathogen is killed. Accordingly, the present invention encompasses polynucleotides encoding such protease cleavage dependent cytotoxic molecules, as well as expression cassettes, vector and cells containing such polynucleotides and polypeptides encoded by such polynucleotides

The nucleic acids of the invention may be used to reduce the viability of a cell expressing a protease encoded by a pathogen, particularly a viral pathogen. These methods may be used as an assay to determine the effectiveness of a nucleotide against a strain of pathogen. The nucleotides of the invention can also be used to reduce the pathogen load of a subject that is infected by a pathogen. As such, the subject methods are particularly useful for the treatment of viral disease such as HIV and HCV, where cells infected by the virus express a viral-encoded protease not native to an uninfected cell.

In further describing the invention, the polynucleotides of the invention will be described first, followed by a description of the polypeptides of the invention and the methods for using the polynucleotides to reduce the viability of a cell expressing a pathogen protease and reduce the pathogen load of a subject infected with a pathogen.

Cleavage-Dependent Cytotoxic Polypeptides

In one aspect, the invention provides a pathogen protease cleavage-dependent cytotoxic polypeptide. In general the polypeptide is a modified version of naturally occurring pro-polypeptide or variant thereof having a pro-domain and a cytotoxic domain. The modified pro-polypeptide contains a sequence-specific protease cleavage site that is not native to the pro-polypeptide, which cleavage site is operably inserted between the pro-domain and the cytotoxic domain. The cytotoxic domain of the modified pro-polypeptide has little or no detectable cytotoxic activity until cleaved from the pro-polypeptide by a pathogen protease, usually a sequence specific pathogen protease. Thus the cytotoxic domain is also referred to herein as a “cleavage dependent cytotoxic polypeptide”.

In general, the subject polypeptide is a modified version of a naturally occurring pro-polypeptide or a biologically active variant thereof. The pro-polypeptide is usually a pro-polypeptide of a protease, an apoptotic molecule, or a toxin such as a snake, spider, bacterial, plant or fungal toxin. In most embodiments the pro-polypeptide is a precursor for a “mature” polypeptide that has a cytotoxic activity, and, as such, the pro-polypeptide has significantly cytotoxicity if in a cell as compared to the mature polypeptide. In general, a pro-polypeptide exhibits about 20% or less, about 10% or less, about 5% or less, about 2% or less, about 1% or less or even less than about 0.5% of the cytotoxicity of a mature polypeptide when present in a cell, as measured by a standard test human cell viability.

In many embodiments, the pro-polypeptide is a human pro-polypeptide or derived from a human pro-polypeptide, and contains three domains: a pro-domain, a protease site, and a cytotoxic domain. In many embodiments, the pro-polypeptide is involved in the cellular apoptosis pathway (reviewed in Raff, Nature 396, 119-122 (1998), Thornberry et al., Science 281, 1312-1316 (1998), Hengartner, Cell 104, 325-328 (2001) and Fraser et al Cell 85, 781-784. (1996)) such as procaspase-3 (e.g. SEQ ID NO:4), procaspase-8 (e.g. SEQ ID NO:6) or BH3 interacting domain death agonist (BID) pro-polypeptide (SEQ ID NO:1). A modified pro-polypeptide based on BID is of particular interest.

Modification of Endogenous Cleavage Sites

In certain embodiments, the pro-polypeptide is modified so as to inactivate one or more endogenous protease cleavage sites.

Typically, a pro-polypeptide contains at least 1, on many occasions 2 and sometimes 3, 4 or 5 or more endogenous recognition sites for a sequence-specific protease that cleaves the pro-polypeptide into a pro-domain and a cytotoxic domain. These “native” recognition sites can be cleaved by a cellular protease that recognizes the sites. While the identity of the proteases that cleave at these native sites are often unknown, the sequence-specific recognition sites in the pro-polypeptides are often known. For example, BID may be cleaved at any one or more of three internal Asp sites (Asp59, Asp75, and Asp98) to activate the cytotoxic activity of the mature BID polypeptide (Gross et al. (1999) J. Biol. Chem 274: 1156-1163; Li et al. (1998) Cell 94: 491-501).

Native sequence-specific protease recognition sequences for caspases and other molecules in apoptosis are well known (Earnshaw et al. (1999) Annu. Rev. Biochem. 68: 383-424). The LQTD/recognition motif for the predominant p15 fragment formed by cleavage of BID by caspase 8 is similar to the consensus for caspase 8. Li et al. (Cell 94: 491-501) show that LQTD59/is the site for caspase 8 cleavage and and IEAD75/is the cleavage site for granzyme B The consensus for capases 6, 8, 9 is (I/L/V)EXD where X is any amino acid. The consensus motif for caspase 2, 3, 7 is DEXD, where X is any amino acid. The sequence DEMD is the classic recognition site of caspase 3, but DQMD is also cleaved very well. Cleavage at D59 yields the predominant p15 product while further cleavage at D75 and D98 yield the minor pl3 and pl 1 fragments, respectively (Gross et al. J. Biol. Chem. 274: 1156-1163). Recognition sequences for granzyme B are also known in the art (e.g. Thomas et al Proc. Natl. Acad. Sci. 98: 14985-14990, 2001 and Barry et al., Mol Cell Biol 20:3781, 2000). As such, native sequence-specific protease recognition sequences may be identified in a pro-polypeptide by comparing the sequence of the pro-polypeptide to known protease recognition sequences.

Native sequence-specific protease recognition sequences may also be identified empirically. For example, since proteases involved in apoptosis, such as granzyme B and certain caspases, cleave at an Asp site, a series of modified pro-polypeptides may be produced that each have a different Asp site modified to e.g. a Glu site and tested for activity using the methods described in the Examples section or other methods known in the art (e.g. Harris et al., J. Biol. Chem. (1998) 273:27346-27373).

Pro-polypeptides modified in a protease recognition site are not cleaved at that site and have reduced toxicity in a cell as compared to an unmodified pro-polypeptide. Alternatively, cleavage sites of a pro-polypeptide may be determined biochemically, e.g. through incubating a pro-polypeptide with a protein extract containing a protease, e.g. granzyme B or a caspase, and determining the C-or N-termini of any resultant peptide fragments of the pro-polypeptide using peptide sequencing.

BID may be cleaved by the granzyme B or caspase 8 proteases and potentially other proteases to activate its pro-apoptotic, cytotoxic activity. A protease site of a pro-polypeptide is within a recognition site for a protease that is typically at least 4 amino acids in length, at least 6 amino acids in length, normally about 7 amino acids in length, and sometimes more than 8, 10, 12 or 14 amino acids in length. In most embodiments, BID is cleaved at a Cys residue. Typically a pro-polypeptide is a human genome-encoded polypeptide or a biological variant thereof.

In a preferred embodiment, a modified pro-polypeptide is altered in at least 1, on many occasions 2 and sometimes 3, 4 or 5 or more recognition sites for a sequence-specific protease that can result in liberation of a mature polypeptide or portion thereof retaining cytotoxic activity in a cell. Usually one of these “native” recognition sites is substituted with a non-native recognition site for a pathogen protease, as described above. Additional native recognition sites may be altered such that they are not cleaved by a cellular protease that recognizes the native recognition site. This can be done by deleting, substituting or adding amino acids in the recognition site. In many embodiments, an amino acid at the site of cleavage is modified. In modified BID for example, the Asp amino acid residues at positions 59, 75, and 98 of the BID amino acid sequence may be substituted with, for example, Glu residues. In many embodiments, the residue at position 98 of the BID amino acid sequence is substituted with, for example, a Glu residue. Any amino acid substitution at these positions will typically cause the site to not become a substrate for a cellular protease specific for the unmodified site. Amino acid alterations may be at positions equivalent to the above positions if BID sequence has been altered (e.g. lengthened or shortened at the N-terminus), as would be recognized by one of skill in the art.

A modified pro-polypeptide altered in at least two native recognition sites for at least one sequence specific protease is usually less toxic in a cell than an equivalent modified pro-polypeptide altered in a single native recognition sites. As such, in most embodiments, a modified pro-polypeptide altered in at least two native recognition sites is more than 95% less, more than 90% less, more than 75% less, more than 50% less, more than 35% less or more than 20% less toxic in a cell than an equivalent modified pro-polypeptide altered in a single native recognition site, as measured by a standard test human cell viability.

Modification to Include Non-Endogenous, Pathogen-Specific Protease Cleavage Site

The subject modified pro-polypeptide is typically a pro-polypeptide that is modified to operably include a sequence specific protease site that is not native to the pro-polypeptide. The choice of which “non-native” protease site to be included depends on the pathogen to which the system will be used with. For example, a pro-polypeptide (or its encoding polynucleotide) to be used to treat an HCV infection is engineered to have a HCV protease site (e.g. the NS3 serine protease), whereas a pro-polypeptide (or its encoding polynucleotide) to be used to treat an HIV-1 infection will usually be engineered to have a HIV-1 protease recognition site. Furthermore, the protease cleavage site may be further selected depending upon the genotype of the infecting pathogen (e.g., for HCV, genotypes 1, 2, 3 and the like; for HIV, genotypes HIV-1, HIV-2, and the like). Exemplary HCV protease sites are as follows: DLEVVT/STWV (NS3/NS4A cis cleavage site; SEQ ID NO:8), DEMEEC/ASHL (NS4A/NS4B trans cleavage site; SEQ ID NO:9), DCSTPC/SGSW (NS4B/NS5A trans cleavage site; SEQ ID NO:10) and EDVVCC/SMSY (NS5A/NS5B trans cleavage site; SEQ ID NO:11), where “/” is a cleavage site, the residue immediately preceding the cleavage site is usually a Cys but can be a Thr, the residue that is first in the site is an Asp but can be a Glu, and the residue at the four position after the cleavage site is a hydrophobic amino acid such as Val, Leu, Ala, Trp or Tyr. The conserved residues at first in the site and immediately preceding the cleavage site are usually present in a HCV protease cleavage site. Cleavage sites for the HCV proteases, in particular protease NS3, are further described in Grakoui, A. et al. (Journal of Virology 67: 2832-2843, 1993) and Urbani et al. (Journal of Biological Chemistry 272: 9204-9209 (1997). Table 1 shows an exemplary list of protease sites that may be engineered into a pro-polypeptide to make a modified pro-polypeptide, where “- - -” is the cleavage site. It is understood that these sequences maybe shortened. In many embodiments, the protease cleavage site for a vector for use with HCV is AEDVVCCSMSYS (SEQ ID NO:20), the protease cleavage site for use with HIV-1 is SQVSQNYPIVQNLQ (SEQ ID NO:21) or CTERQANFLGKIWP (SEQ ID NO:22).

TABLE 1

| PATHOGEN | PROTEIN | SEQUENCE |
|---|---|---|
| HIV-1 | GAG PROTEINS | |
| | p17-p24 | SQVSQNY---PIVQNLQ |
| | | (SEQ ID NO:12) |
| | p7-p1 | CTERQAN---FLGKIWP |
| | | (SEQ ID NO:13) |
| | POL PROTEINS | |
| | P6′-Protease | GTVSFSF---PQITLWQ |
| | | (SEQ ID NO:14) |
| | Protease-RT | IGCTLNF---PISPIET |
| | | (SEQ ID NO:15) |
| HCV | NS3-NS4A | CMSADLWVVT--STWVLVGGVL |
| | | (SEQ ID NO:16) |
| | NS4A-NS4B | YQEFDEMEEC---ASHLPYIEQG |
| | | (SEQ ID NO:17) |
| | NS4B-NS5A | WISSECTTPC---SGSWLRDIWD |
| | | (SEQ ID NO:18) |
| | NS5A-NS5B | GADTEDVVCC--SMSYTWTGAL |
| | | (SEQ ID NO:19) |

As such, the pro-polypeptide may be designed for use with any RNA virus, or other intracellular pathogen, as long as the sequence specificity for a protease of the pathogen is known or readily identifiable.

Additional pathogen-specific proteases and specified cleavage sites have been described and can be used in accord with the present invention.

For example, an HSV-1 maturational protease and protease cleavage site has been described. See e.g. Hall, M. R. T. and W. Gibson, Virology, 227:160 (1997); the disclosure of which is incorporated by reference. Further, two aspartic proteinases referenced as plasmepsins I and II have been found in the digestive vacuole of P. falciparum. The corresponding proteinase cleavage sites have also been disclosed. See e.g., Moon, R. P., Eur. J. Biochem., 244:552 (1997). Further pathogen-specific protease cleavage sites include the HSV-1 protease cleavage sites p17-p24 (SQVSQNY-PIVQNLQ; SEQ ID NO:23), p7-p1(CTERQAN-FLGKIWP; SEQ ID NO:24), and pr-RT (IGCTLNF-PISPIET; SEQ ID NO:25).

The ordinarily skilled artisan will appreciate that any of the above-referenced protease cleavage sites can be modified as desired (e.g., by site-specific mutagenesis) so long as the sites are specifically cleaved by the pathogen-specific protease for which they are intended. In some embodiments, it maybe desirable to use the minimal amino acid sequence necessary for specific proteolytic cleavage, e.g., to optimize size of modified pro-polypeptide and its encoding nucleic acid. Minimal cleavage site sequences have been reported for many pathogen-specific protease cleavage sites. Alternatively, the minimal sequence for a desired proteolytic cleavage site can be readily obtained by mutagenesis, particularly deletion analysis and site specific mutagenesis (e.g., alanine scanning mutagenesis). The modified cleavage site can be readily assayed in a standard protease cleavage assay as described below.

“Specifically cleaved” as used herein means that peptide bonds in a specified protease cleavage site are specifically broken (i.e. hydrolyzed) by a protease that specifically binds the amino acid sequence of the protease cleavage site. Specific cleavage of a protease cleavage site can be assayed according to a variety of techniques, e.g., analysis of protein fragments, competitive inhibition of protease activity, and the like.

The pathogen protease may be a serine endopeptidase, a cysteine endopeptidase, an aspartic endopeptidase, a metalloendopeptidases or an endopeptidases of unknown catalytic mechanism. In many embodiments the protease site is a site for a viral sequence specific protease. In an embodiment of particular interest, the protease site may be chosen from cytomegalovirus, herpes simplex virus type-1, hepatitis C virus (HCV, including HCV genotypes 1, 2, 3, and the like), human immunodeficiency virus type 1, human immunodeficiency virus type 2 and Kaposi's syndrome associated herpes virus protease cleavage sites, picornaviruses (foot and mouth disease virus, polio virus, coxsackievirus), flaviviruses (West Nile fever, Yellow fever, Dengue fever, Japan encephalitis, Murray Valley encephalitis, St. Louis encephalitis), togarviruses (Eastern equine encephalitis, Western equine encephalitis, Venezuelan encephalitis, Chikungunya, rubella, Ross River fever), caliciviruses (Norwalk agent), adenoviruses although several other pathogens and corresponding protease sites are known in the art. Protease recognition sequences (i.e. protease sites) of the modified pro-polypeptides of the invention are usually at least 6 amino acids in length, at least 8 amino acids in length, at least 10 amino acids in length and often at least 12 or 14 or more amino acids in length.

Usually, the non-native protease recognition sequences are inserted into the a pro-polypeptide sequences by replacing amino acids of the pro-polypeptide, by inserting the non-native recognition sequences between two amino-acid residues of the pro-polypeptide, or by a combination of these methods. In many embodiments, the non-native protease recognition sequences replace or partially replace the native recognition sequence that provides for release of the pro-domain and mature polypeptide upon cleavage. Regardless of the method of introduction of the non-native, pathogen-specific protease cleavage site, the native cleavage site present in the starting pro-polypeptide is altered so that in the modified pro-polypeptide it is not longer cleaved by the cellular protease that recognizes the unmodified native recognition sequence.

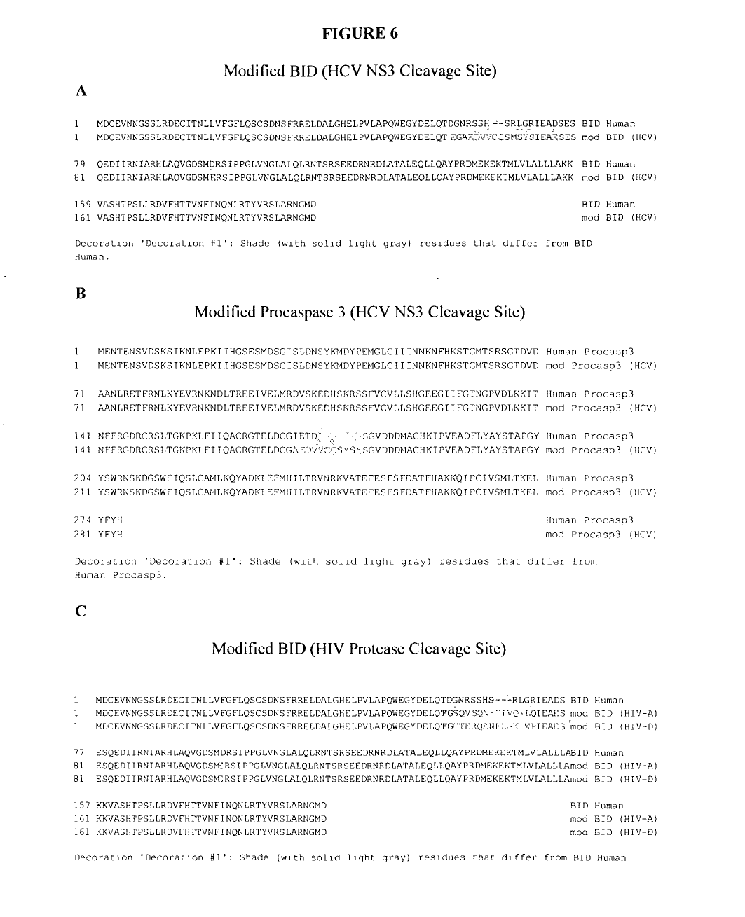

Exemplary modified pro-caspase-3 (SEQ ID NO:5), pro-caspase-8 (SEQ ID NO:7) and pro-BID pro-polypeptides (SEQ ID NOS:2 and 3) with HIV or HCV protease sites and modified endogenous protease cleavage sites are shown in FIG. 6.

Nucleic Acids Encoding a Modified Pro-Polypeptide

The invention provides a nucleic acid comprising a nucleotide sequence encoding a modified pro-polypeptide (also referred to herein as a “pathogen protease cleavage-dependent cytotoxic polypeptide”, as described above. Since the genetic code and recombinant techniques for manipulating nucleic acid are known, and the polypeptide sequence a modified pro-polypeptide is described as above, the design and production of these nucleic acids is well within the skill of an artisan.

The subject nucleic acids usually comprise an single open reading frame encoding the subject modified pro-polypeptide, however, in certain embodiments, since the target cell for expression of the pro-polypeptide may be a eukaryotic cell, e.g., a human cell, the open reading frame may be interrupted by introns. Subject nucleic acid are typically part of a transcriptional unit which contains, in addition to the nucleic acid a 3′ and a 5′ untranslated regions (UTRs) which may direct RNA stability, translational efficiency, etc. The subject nucleic acid may also be part of an expression cassette which contains, in addition to the nucleic acid a promoter, which directs the transcription and expression of a pro-polypeptide-encoding RNA, and a transcriptional terminator. In some embodiments, the promoter is inducible by the pathogen, with which the pro-polypeptide is to be used.

Exemplary eukaryotic promoters include, but are not limited to, the following: the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-310, 1981); the yeast gall gene sequence promoter (Johnston et al., Proc. Natl. Acad. Sci. (USA) 79:6971-6975, 1982); Silver et al., Proc. Natl. Acad. Sci. (USA) 81:5951-59SS, 1984), the CMV promoter, the EF-1 promoter, Ecdysone-responsive promoter(s), tetracycline-responsive promoter, and the like. Viral promoters may be of particular interest as they are generally particularly strong promoters. In certain embodiments, a promoter is used that is a promoter of the target pathogen. Promoters for use in the present invention are selected such that they are functional in the cell type (and/or animal) into which they are being introduced.

In some embodiments, where the intracellular pathogen is a virus, the promoter is a viral promoter that is derived from or activatable by one or more viral transcription factors of the infecting viral pathogen. For example, where the infecting virus is HIV, the promoter to drive expression of the modified pro-polypeptide of the invention is an HIV promoter or other promoter from which transcription is increased in a host cell infected with HIV.

The subject nucleic acid segments may also contain restriction sites, multiple cloning sites, primer binding sites, ligatable ends, recombination sites etc., usually in order to facilitate the construction of a nucleic acid encoding a modified pro-polypeptide.

In most embodiments, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) is used to substitute, delete, and/or add appropriate nucleotides in the nucleic acid sequence encoding a parental antibody framework-coding sequence in order to create a modified pro-polypeptide-encoding nucleic acid from an unmodified pro-polypeptide-encoding nucleic acid.

For example, site directed mutagenesis may be used to introduce/delete/substitute nucleic acid residues in the polynucleotide encoding a pro-polypeptide native protease site such that the mutagenized polynucleotide encodes a pro-polypeptide non-native protease site. In other methods, PCR is used. One PCR method utilizes “overlapping extension PCR” (Hayashi et al., Biotechniques. 1994: 312, 314-5) to create modified pro-polypeptide-encoding sequences. In this method, the nucleic acid residue codons encoding the substituted/inserted/deleted amino acid residues in the modified pro-polypeptide are engineered into PCR primers. Multiple overlapping PCR reactions using the parental nucleic acid sequence as a template generates a modified nucleic acid. The product of many of these methods is a modified framework region. Several other methods for modifying nucleic acids may also be employed, including the “Quickchange”™ Kit of Stratagene (La Jolla, Calif.).

The invention further provides vectors (also referred to as “constructs”) comprising a subject nucleic acid. In many embodiments of the invention, nucleic acid sequences encoding a modified pro-polypeptide will be expressed in a host after the sequences have been operably linked to an expression control sequence, including, e.g. a promoter. The subject nucleic acids are also typically placed in an expression vector that can replicable in a host organisms either as an episome or as an integral part of the host chromosomal DNA. Commonly, expression vectors will contain selection markers, e.g., tetracycline or neomycin, to permit detection of those cells transformed with the desired DNA sequences (see, e.g., U.S. Pat. No. 4,704,362, which is incorporated herein by reference). Vectors, including single and dual expression cassette vectors are well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Suitable vectors include viral vectors, plasmids, cosmids, artificial chromosomes (human artificial chromosomes, bacterial artificial chromosomes, yeast artificial chromosomes, etc.), mini-chromosomes, and the like. Retroviral, adenoviral and adeno-associated viral vectors are usually used.

Host Cells

The invention further provides host cells, including isolated in vitro host cells (e.g., for construct production and/or use in screening assays) and in vivo host cells of a non-human animal, that comprise a nucleic acid or a vector of the invention.

E. coli is a prokaryotic host useful for cloning the nucleic acid sequences of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, may also be used for expression. Saccharomyces or Picha are preferred hosts, with suitable vectors having expression control sequences, such as promoters, including the 3-phosphoglycerate kinase promoter or those of other glycolytic enzyme genes, and an origin of replication, termination sequences and the like as desired.

In addition to microorganisms, mammalian tissue cell culture may also be used to express and produce the polypeptides of the present invention (see, Winnacker, “From Genes to Clones,” VCH Publishers, New York, N.Y. (1987), which is incorporated herein by reference). Eukaryotic cells are preferred, because a number of suitable host cell lines capable of secreting intact antibodies have been developed in the art, and include the CHO cell lines, various COS cell lines, HeLa cells, preferably myeloma cell lines, etc, and transformed B-cells or hybridomas. Expression vectors for these cells can include expression control sequences, such as an origin of replication, a promoter, an enhancer (Queen et al., Immunol. Rev., 89, 49-68 (1986), which is incorporated herein by reference), and necessary processing information sites, such as ribosome binding sites, RNA splice sites, polyadenylation sites, and transcriptional terminator sequences. Preferred expression control sequences are promoters derived from immunoglobulin genes, SV40, Adenovirus, cytomegalovirus, Bovine Papilloma Virus, and the like. Of particular interest are mammalian cells, including primary cells and cell lines, that can support infection by an intracellular pathogen for which the modified pro-polypeptide is specific (i.e., the modified pro-polypeptide contains a protease cleavage site for a protease produced by the intracellular pathogen).

The subject modified pro-polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, may be used as the expression host cells. In some situations, it is desirable to express the gene in eukaryotic cells, where the encoded protein will benefit from native folding and post-translational modifications. Polypeptides can also be synthesized in the laboratory. Polypeptides that are subsets of the complete sequences of the subject proteins may be used to identify and investigate parts of the protein important for function.

In embodiments of particular interest, the host cell is a mammalian (e.g.) human cell that may be infected with an intracellular pathogen such as a virus and that is susceptible to the cytotoxicity of the cytotoxic domain cleavage product of the pro-polypeptide. In certain embodiments the human cell is a CHOP cell, a huh7 cell or a H9C2 cell, and the cell is chosen because it is susceptible to infection by a virus of interest.

Screening Methods using Modified Pro-Polypeptides

The invention also provides methods of screening modified pro-polypeptides. In many embodiments, these methods are in vitro methods, involving introducing a subject polypeptide or a nucleotide encoding a subject pro-polypeptide in a cell expressing a pathogen-encoded protease, incubating the cell under conditions that allow for expression of said pro-polypeptide and determining the viability of the cell. In other embodiments, these methods are in vivo methods, involving introducing a subject polypeptide or a nucleotide encoding a subject pro-polypeptide in a non-human animal infected with a pathogen expressing a pathogen-encoded protease, and determining the symptoms of the animal.

In Vitro Assays

In general, this method involves introducing a nucleic acid encoding a subject modified pro-polypeptide containing a recognition site for a pathogen protease into the cell expressing a pathogen protease, incubating the cell to allow for expression of said pro-polypeptide and determining the viability of the cell.

In most embodiments, up to 20%, up to 50%, up to 70%, up to 80%, up to 90%, up to 95%, up to 98%, and even up to 99% or 99.5% of cells expressing a pathogen-encoded protease have reduced viability (e.g. are killed) by this method. The viability of a cell is usually determined by whether or not it is living, and this can be measured using a standard viability test such as a test for the uptake of a dye such a trypan blue or a fluor.

In general, the protease that cleaves the protease cleavage site of the modified pro-polypeptide is present in a cell as a result of the presence of an intracellular pathogen (i.e., the cell is infected with an intracellular pathogen). In many embodiments, however, the target cell is recombinant for the protease of interest (e.g., as a result of infection with a native or recombinant virus, the presence of a viral replicon, plasmid or other recombinant molecule that is capable of expressing a protease in a cell).

In many embodiments, the pathogen-encoded protease is a protease of an intracellular pathogen such as a virus. Suitable viral pathogens may be found in Virus Taxonomy. The Classification and Nomenclature of Viruses, The Seventh Report of the International Committee on Taxonomy of Viruses, (van Regenmortel et at Eds (2000). Academic Press, SanDiego) or found at the The Universal Virus Database (version 2) found at the worldwide website of the Australian National University. Cells expressing a protease of an RNA virus, in particular a (+) RNA virus such as HCV, a (−) RNA virus and an RNA-RT virus, such as HIV-1, are of interest.

In general, a nucleic acid encoding a subject pro-polypeptide is introduced into a cell containing a pathogen protease using methods known in the art (e.g. viral infection, transfection, electroporation and the like, discussed in Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) and the cell is incubated for a period of 1 hour, 12 hours, 24 hours or even several days until the pro-polypeptide is expressed.

In many embodiments, the subject methods of reducing the viability of a target cell expressing a protease encoded by a pathogen are used in assays for determining the effect of a modified pro-polypeptide on the viability of a cell expressing a pathogen-encoded protease. In general, the cell is incubated for more than about 1 hour, 3 hours, 8 hours, 24 hours, or even 48 hours or more before its viability is measured. The viability of a cell is determined by whether or not it is living, and this can be measured using a standard viability test such as a test for the uptake of a dye such a trypan blue or a fluor. In most embodiments the assay is performed on a plurality of cells expressing a pathogen protease, and cell viability is expressed as a percentage. In most embodiments, up to 20%, up to 50%, up to 70%, up to 80%, up to 90%, up to 95%, up to 98%, and even up to 99% or 99.5% of cells expressing a pathogen-encoded protease have reduced viability (e.g. are killed) in this assay as compared to controls that are administered an unmodified pro-polypeptide. Such methods usually reduce the viability of the subject cell, and, may be used in a variety of assays.

The subject methods may be used to individually test a plurality of different pro-polypeptides for their effectiveness against a protease encoded by a particular pathogen, e.g. a strain of a virus, to determine the most effective pro-polypeptide for reducing the viability of a cell expressing a protease encoded by the strain.

In other embodiments, the subject methods may also be used to individually test a plurality of different proteases encoded by a plurality of different pathogens (e.g. strains of a virus) to determine whether they are susceptible to a particular pro-polypeptide.

In other embodiments, the subject assays may be used to individually test a plurality of agents, such as small molecules, peptide inhibitors and the like to determine whether they inhibit a pathogen-encoded protease. In these assays, a test agent may be co-introduced with a modified pro-polypeptide into a pathogen protease expressing cell and the viability of the cell determined. In many embodiments, a decrease of cell viability of more than 10%, more than 30%, more than 50%, more than 70% or even more than 80% or more than 90% or more as compared to a control not administered a test agent indicates that the agent inhibits the pathogen-encoded protease. Agents that increase cell viability may be used in the treatment of a virally infected subject.

In some embodiments of the invention a subject nucleic acid is administered at the same time as or after a pathogen protease-encoding nucleic acid (e.g. a virus, recombinant virus, protease-encoding plasmid etc.) is administered to a cell. However, in certain other embodiments, the subject nucleic acid encoding a pro-polypeptide is administered before the pathogen protease-encoding nucleic acid. In these embodiments, the subject nucleic acids may increase the viability of a cell and provide “protection” of a cell against a future viral infection.

In Vivo Assays

In general, this method involves, for example, administering a nucleic acid encoding a modified pro-polypeptide containing a recognition site for a pathogen protease into a non-human animal model infected with a human pathogen. In this context, the non-human animal model is usually infected with a human pathogen that expresses a site specific protease, such as a viral pathogen. Many such animal models using mammals, especially of mouse, monkeys. rats, cats, dogs, guinea pigs, etc., are known to one of skill in the art. Mouse models, in particular the mouse models for HCV (described in PCT publication WO 01678) and HIV infection (described in U.S. Pat. No. 5,612,018) are of interest.

In most embodiments, upon administration of a subject nucleic acid, a symptom (e.g. viability of pathogen infected cells, lesions, bleeding, bruising, titer, the number of infected cells) of the pathogen exhibited by the animal is reduced up to 20%, up to 50%, up to 70%, up to 80%, up to 90%, up to 95%, up to 98%, and even up to 99% or 99.5% as compared to an animal that is not administered a subject nucleic acid. In other embodiments, upon administration of a subject nucleic acid, a symptom (e.g. CD4 count, ALT or HAAT activity, etc.) of the pathogen exhibited by the animal is increased up to 20%, up to 50%, up to 70%, up to 80%, up to 90%, up to 95%, up to 98%, and even up to 99% or 99.5%, as compared to an animal that is not administered a subject nucleic acid.

In many embodiment, a blood sample is taken from the animal and tested for the level of a blood product, such as a virus, cell, a protein, or a molecule (e.g. viral titer, viral genome, viral mRNA, CD4 count, ALT or HAAT activity etc.). In other embodiments, a sample of tissue is taken from the test animal and symptoms (e.g. cell death, lesions, viral titer etc) are measured.

Treatment of Infection of a Subject by an Intracellular Pathogen

The invention also provides a method of treating an intracellular pathogen infection in a subject. In this context, treatment can involve reduction of pathogen load in the infected subject (e.g., reduction of viral load or viral titer). The invention also contemplates preventing or reducing the risk of symptoms or disease of infection by an intracellular pathogen in a susceptible subject. Examples of subjects in this latter category include, but are not necessarily limited to, organ transplant recipients (e.g., liver transplants, bone marrow or other immune cell transplants, and the like). Of particular interest is treatment of a subject having a chronic HCV infection, including those undergoing liver transplant as therapy so as to clear the HCV infection and reduce the risk of re-infection of the donor liver and immunocompromised or otherwise immune deficient subjects (e.g., due to autoimmune disease, AIDS, genetic defect, and the like).

In one embodiment of particular interest, the invention involves administering a subject nucleic acid encoding a pro-polypeptide a subject infected with the pathogen. In another embodiment, the modified pro-polypeptide is delivered. In this latter embodiment, the modified pro-polypeptide may necessitate further modification or formulation to facilitate intracellular delivery of the polypeptide to the infected host cell. Methods for accomplishing intracellular delivery of a protein of interest are known in the art, see, e.g., U.S. Pat. No. 6,221,355.

The modified pro-polypeptide for use in therapy is selected according to the infecting intracellular pathogen. In most embodiments, the pathogen is a pathogen with an encoded sequence specific protease that is expressed in host cells infected by the pathogen. In many embodiments the pathogen is an intracellular pathogen, e.g, a virus, particularly a RNA virus. As such, the methods may be useful for reducing pathogen load of a subject infected by any pathogen that expresses a protease in the host. Suitable viral pathogens may be found in Virus Taxonomy: The Classification and Nomenclature of Viruses. The Seventh Report of the International Committee on Taxonomy of Viruses, (van Regenmortel et at Eds (2000) Academic Press, San Diego). In many embodiments, the subject methods are used for HIV and HCV.

Specific exemplary intracellular pathogen infections contemplated for treatment according to the invention include, but are not necessarily limited to, those infection associated with hepatitis C virus (HCV, including HCV genotypes 1, 2, 3, and the like), human immunodeficiency virus (HIV, including HIV-1 and HIV-2, and the like), cytomegalovirus, herpes simplex virus (HSV, including type-I and type 2), Kaposi's syndrome associated herpes virus, human T cell lymphotropic virus (HTLV, including. HTLV-I and HTLV-II, yellow fever virus, certain flaviviruses (e.g., Ebola virus), rhinoviruses, and the like. In addition, the pathogen can be any one of those capable of causing malaria or a medical condition relating to same such as P. falciparum, P. vivax, P. ovale, or P. malariae.

The invention also provides methods of treatment of other diseases caused by or otherwise associated with a viruses such as picornaviruses (foot and mouth disease virus, polio virus, coxsackievirus), flaviviruses (West Nile fever, Yellow fever, Dengue fever, Japan encephalitis, Murray Valley encephalitis, St. Louis encephalitis), togarviruses (Eastern equine encephalitis, Western equine encephalitis, Venezuelan encephalitis, Chikungunya, rubella, Ross River fever), caliciviruses (Norwalk agent), adenoviruses (influenza viruses do not encode their own viral protease) as well as diseases associated with viruses of the herpes family, e.g., herpes simplex viruses (HSV) including herpes simplex 1 and 2 viruses (HSV 1, HSV 2), varicella zoster virus (VZV; shingles), human herpes virus 6, cytomegalovirus (CMV), Epstein-Barr virus (EBV), and other herpes virus infections, such as feline herpes virus infections, and diseases associated with hepatitis viruses including hepatitis C viruses (HCV). Examples of clinical conditions which are caused by such viruses include herpetic keratitis, herpetic encephalitis, cold sores and genital infections (caused by herpes simplex), chicken pox and shingles (caused by varicella zoster) and CMV-pneumonia and retinitis, particularly in immunocompromised patients including renal and bone marrow transplant patients and patients with Acquired Immune Deficiency Syndrome (AIDS). Epstein-Barr virus can cause infectious mononucleosis, and is also suggested as the causative agent of nasopharyngeal cancer, immunoblastic lymphoma and Burkitt's lymphoma.

The pathogen may be present in a virulent, latent, or attenuated form, or in a combination of those forms. The subject may be symptomatic or asymptomatic. In addition, where the subject has or is susceptible to infection by one or more intracellular pathogens, the invention contemplates administration of one or more modified pro-polypeptides corresponding to the relevant pathogens. Where multiple modified pro-polypeptides are to be administered, they may be administered separately, simultaneously, in the same or different formulations, or, where a polynucleotides (usually DNA) encoding the modified pro-polypeptide is administered, in the same or different nucleic acid molecules.

Although the dosage used will vary depending on the clinical goals to be achieved, a suitable dosage range is one which provides up to about 1 μg to about 1,000 μg or about 10,000 μg of a subject pro-polypeptide-encoding nucleic acid that reduces pathogen load can be administered in a single dose. Alternatively, a target dosage of an subject pro-polypeptide-encoding nucleic acid that reduces pathogen load can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

A subject modified pro-polypeptide or modified pro-polypeptide-encoding nucleic acid that reduces pathogen load is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intratumoral, subcutaneous, intradermal, topical application, intravenous, rectal, nasal, oral and other parenteral routes of administration. Routes of administration may be combined, if desired, or adjusted depending upon the agent and/or the desired effect. The composition can be administered in a single dose or in multiple doses.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

Methods of administration of the agent through the skin or mucosa include, but are not necessarily limited to, topical application of a suitable pharmaceutical preparation (see., e.g., U.S. Pat. No. 6,087,341 for delivery of nucleic acid to the skin), transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of several days or more.

In many embodiments, the subject nucleic acids are administered to a subject systemically, e.g. through administration into the bloodstream, or locally, through injection directly into or near to a target organ. Local administrations usually depend on the location of the virus infection, and may include renal subcapsular, subcutaneous, central nervous system (including intrathecal), intravascular, intrahepatic, intrasplenic, intrasplanchnic, intraperitoneal (including intraomental), or intramuscular administrations. In some embodiments, the administrations are directly into the hepatic duct, or into lymph nodes, bone marrow, and other organs of the body. In many embodiments, administration is usually intravenous, intraportal, intrasplanchnic, into the portal vein or hepatic artery of a donor liver prior to transplant (i.e. administration is carried out ex-vivo), or in vivo after revascularization at the time of transplant. In some embodiments, particularly when the compositions of the invention are administered to an animal, a modified pro-polypeptide is administered by injection directly into the jugular vein or the portal vein of an infected subject.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, pathogen load (e.g. pathogen titre). As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

A subject polynucleotide can be delivered as a naked -polynucleotide, or associated with (“complexed with”) a delivery vehicle. “Associated with”, or “complexed with”, encompasses both covalent and non-covalent interaction of a polynucleotide with a given delivery vehicle.

In certain embodiments it is preferable to use a vector that does not permit the transmission of introduced nucleic acids through the germ-line of the host. In these embodiments, a vector that does not integrate into a genome of the host, e.g. a non-integrative vector may be used. For example, pro-polypeptide nucleic acids may be delivered by liposome encapsulated non-integrative expression vector using a CMV promoter to drive the pro-polypeptide may be used that drives modified BID is also an option. Also, a non-integrative vector may be introduced as naked DNA into the hepatic portal vein and would be taken up by hepatocytes. In certain embodiments, administration of the vector directly into a target organ (e.g. a liver, lymph node etc.) or near, into a duct emptying into, or a blood vessel going into a target organ is preferred.

Viral Delivery Vehicles

A subject polynucleotide can be associated with viral delivery vehicles. As used herein, a “viral delivery vehicle” intends that the polynucleotide to be delivered is encapsidated in a viral particle.

Numerous viral genomes useful in in vivo transformation and gene therapy are known in the art, or can be readily constructed given the skill and knowledge in the art. Included are replication competent, replication deficient, and replication conditional viruses. Viral vectors include adenovirus, mumps virus, a retrovirus, adeno-associated virus, herpes simplex virus (HSV), cytomegalovirus (CMV), vaccinia virus, and poliovirus, and non-replicative mutants/variants of the foregoing. In some embodiments, a replication-deficient virus is capable of infecting slowly replicating and/or terminally differentiated cells, since the respiratory tract is primarily composed of these cell types. For example, adenovirus efficiently infects slowly replicating and/or terminally differentiated cells. In some embodiments, the viral genome itself, or a protein on the viral surface, is specific or substantially specific for cells of the targeted cell. A viral genome can be designed to be target cell-specific by inclusion of cell type-specific promoters and/or enhancers operably linked to a gene(s) essential for viral replication.

Where a replication-deficient virus is used as the viral genome, the production of virus particles containing either DNA or RNA corresponding to the polynucleotide of interest can be produced by introducing the viral construct into a recombinant cell line which provides the missing components essential for viral replication and/or production. Preferably, transformation of the recombinant cell line with the recombinant viral genome will not result in production of replication-competent viruses, e g., by homologous recombination of the viral sequences of the recombinant cell line into the introduced viral genome. Methods for production of replication-deficient viral particles containing a nucleic acid of interest are well known in the art and are described in, for example, Rosenfeld et al., Science 252:431-434, 1991 and Rosenfeld et al., Cell 68:143-155, 1992 (adenovirus); U.S. Pat. No. 5,139,941 (adeno-associated virus); U.S. Pat. No. 4,861,719 (retrovirus); and U.S. Pat. No. 5,356,806 (vaccinia virus). Methods and materials for manipulation of the mumps virus genome, characterization of mumps virus genes responsible for viral fusion and viral replication, and the structure and sequence of the mumps viral genome are described in Tanabayashi et al., J. Virol. 67:2928-2931, 1993; Takeuchi et al., Archiv. Virol., 128:177-183, 1993; Tanabayashi et al., Virol. 187:801-804, 1992; Kawano et al., Virol., 179:857-861, 1990; Elango et al., J. Gen. Virol. 69:2893-28900, 1988.

Non-Viral Delivery Vehicles

A subject polynucleotide can be administered using a non-viral delivery vehicles. “Non-viral delivery vehicle” (also referred to herein as “non-viral vector”) as used herein is meant to include chemical formulations containing naked or condensed polynucleotides (e.g, a formulation of polynucleotides and cationic compounds (e.g., dextran sulfate)), and naked or condensed polynucleotides mixed with an adjuvant such as a viral particle (i.e., the polynucleotide of interest is not contained within the viral particle, but the transforming formulation is composed of both naked polynucleotides and viral particles (e.g., adenovirus particles) (see, e.g., Curiel et al. 1992 Am. J. Respir. Cell Mol. Biol. 6:247-52)). Thus “non-viral delivery vehicle” can include vectors composed of polynucleotides plus viral particles 1.5 where the viral particles do not contain the polynucleotide of interest.

“Non-viral delivery vehicles” include bacterial plasmids, viral genomes or portions thereof, wherein the polynucleotide to be delivered is not encapsidated or contained within a viral particle, and constricts comprising portions of viral genomes and portions of bacterial plasmids and/or bacteriophages. The term also encompasses natural and synthetic polymers and co-polymers. The term further encompasses lipid-based vehicles. Lipid-based vehicles include cationic liposomes such as disclosed by Felgner et al (U.S. Pat. Nos. 5,264,618 and 5,459,127; PNAS 84:7413-7417, 1987; Annals N.Y. Acad. Sci. 772:126-139, 1995); they may also consist of neutral or negatively charged phospholipids or mixtures thereof including artificial viral envelopes as disclosed by Schreier et al. (U.S. Pat. Nos. 5,252,348 and 5,766,625).

Non-viral delivery vehicles include polymer-based carriers. Polymer-based carriers may include natural and synthetic polymers and co-polymers. Preferably, the polymers are biodegradable, or can be readily eliminated from the subject. Naturally occurring polymers include polypeptides and polysaccharides. Synthetic polymers include, but are not limited to, polylysines, and polyethyleneimines (PEI; Boussif et al., PNAS 92:7297-7301, 1995) which molecules can also serve as condensing agents. These carriers may be dissolved, dispersed or suspended in a dispersion liquid such as water, ethanol, saline solutions and mixtures thereof. A wide variety of synthetic polymers are known in the art and can be used.

“Non-viral delivery vehicles” further include bacteria. The use of various bacteria as delivery vehicles for polynucleotides has been described. Any known bacterium can be used as a delivery vehicle, including, but not limited to non-pathogenic strains of Staphylococcus, Salmonella, and the like.

The polynucleotide to be delivered can be formulated as a DNA- or RNA-liposome complex formulation. Such complexes comprise a mixture of lipids which bind to genetic material (DNA or RNA) by means of cationic charge (electrostatic interaction). Cationic liposomes which may be used in the present invention include 3p-[N-(N′, N′-dimethylaminoethane)-carbamoyl]-cholesterol (DC-Chol), 1,2-bis(oleoyloxy-3-trimethylammonio-propane (DOTAP) (see, for example, WO 98/07408), lysinylphosphatidylethanolamine (L-PE), lipopolyamines such as lipospermine, N-(2-hydroxyethyl)-N,N-dimethyl-2,3-bis(dodecyloxy)-1-propanaminium bromide, dimethyl dioctadecyl ammonium bromide (DDAB), dioleoylphosphatidyl ethanolamine (DOPE), dioleoylphosphatidyl choline (DOPC), N(1,2,3-dioleyloxy) propyl-N,N,N-triethylammonium (DOTMA), DOSPA, DMRIE, GL-67, GL-89, Lipofectin, and Lipofectamine (Thiery et al. (1997) Gene Ther. 4:226-237; Felgner et al., Annals N.Y. Acad. Sci. 772:126-139, 1995; Eastman et al., Hum. Gene Ther. 8:765-773, 1997). Polynucleotide/lipid formulations described in U.S. Pat. No. 5,858,784 can also be used in the methods described herein. Many of these lipids are commercially available from, for example, Boehringer-Mannheim, and Avanti Polar Lipids (Birmingham, Ala.). Also encompassed are the cationic phospholipids found in U.S. Pat. Nos. 5,264,618, 5,223,263 and 5,459,127. Other suitable phospholipids which may be used include phosphatidylcholine, phosphatidylserine, phosphatidylethanolamine, sphingomyelin, phosphatidylinositol, and the like. Cholesterol may also be included.

The modified pro-polypeptides of the invention can be administered as a sole active agent, in combination with one or more other modified pro-polypeptides as provided herein, and/or in combination with other medicaments, such as, for example, ribavirn and/or ribavirin derivatives, IFN-α (e.g., IFN-α2a, IFN-α2b, PEG-IFN-α2a, PEG-IFN-α2b, consensus IFN), reverse transcriptase inhibitors (e.g., a dideoxynucleoside including AZT, ddI, ddC, d4T, 3TO, FTC, DAPD, 1592U89 or CS92); and other agents such as 9-(2-hydroxyethoxymethyl) guanine (acyclovir), ganciclovir or penciclovir, interleukin II, or in conjunction with other immune modulation agents including bone marrow or lymphocyte transplants or other medications such as levamisol or thymosin which would increase lymphocyte numbers and/or function as is appropriate.

Additional medicaments that can be co-administered with one or more modified pro-polypeptides of the invention include standard anti-malarial such as those disclosed in Goodman, G. et al. (1993), The Pharmacological Basis of Therapeutics, 8.sup.th ed. McGraw-Hill Inc. pp. 978-198. Preferred anti-malarial drugs include chloroquine, chloroguanidine, pyrimethamine, mefloquine, primaquame and quinine.

Administration of two or more of the above-referenced agents including the modified pro-polypeptides may be referred to as a “cocktail” or “cocktail” therapy.

In most embodiments, pathogen load (e.g., viral load) may be reduced in the subject by up to 10%, up to 30%, up to 50%, up to 70%, up to 90%, up to 95%, or even up to 99% or 95.5% or more in a subject treated with a subject nucleic acid as compared to a subject prior to treatment, or in a subject not treated. When a the load of a pathogen in a subject is measured, it may be measured from an organ of the subject that is infected by the virus, or more usually blood serum.

Kits

Also provided by the subject invention are kits for practicing the subject methods, as described above. The subject kits at least include one or more of: a nucleic acid encoding a modified pro-polypeptide and a vector containing the same. Other optional components of the kit include: restriction enzymes, control primers and plasmids; buffers, cells etc. The nucleic acids of the kit may also have restrictions sites, multiple cloning sites, primer sites, etc to facilitate their ligation other plasmids. The various components of the kit may be present in separate containers or certain compatible components may be precombined into a single container, as desired. In many embodiments, kits with unit doses of the active agent, e.g. in oral or injectable doses, are provided.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

Materials and Methods

Cell lines. BHK-21 cells were purchased from American Type Culture Collection (Manassis, Va.). HEK-293 cells were supplied by Clontech (Palo Alto, Calif.). H9C2 cells were a gift from Dr. Henry Klamut (Ontario Cancer Institute, Toronto, Canada), CHOP cells came from Dr. James Dennis (Samuel Lunenfeld Research Institute, Toronto, Canada), ecotropic Phoenix packaging 293 cells were supplied by Dr. Garry Nolan (Stanford University, Calif.), and Huh7 cells came from Dr. Stanley Lemon (University of Tex., Galveston, Tex.). The HCV replicon cells and their parental cells (HBI-IOA, HBII1-H27 and Huh7) were developed by Dr. Giovanni Migliaccio (IRBM, Rome, Italy). All cell types were propagated in Dulbecco's minimum essential medium (GIBCO/BRL, Gaithersburg, Md.) supplemented with 10% fetal calf serum. In addition, HBI-10A and HBIII-H27 cells were cultivated in the presence of 800 μg/ml of G418 (GIBCO/BRL, Gaithersburg, Md.).

Antibodies. Monoclonal antibody against human BID was purchased from R&D Systems Inc (Minneapolis, Minn.). Monoclonal antibody against HCV core protein was a generous gift from Dr. Kohara (Tokyo, Japan) and monoclonal antibody against HCV NS3 protein was supplied by Dr. Giovanni Migliaccio (Rome, Italy).

NS3 Protease, Modified Caspases and BID. DNA fragments corresponding to the coding sequence of HCV NS3/NS4A (nt. 3420-nt. 5474) and a single-chain HCV protease (NS4A-NS3) were synthesized by polymerase chain reaction (PCR) from p90/HCV FL-long pU (a gift from Dr. Charles Rice) using primer pairs derived from the 5′ and 3′ ends of the NS3 and NS4A genes. The single chain NS4A-NS3 version of the serine protease was constructed as previously reported⁴⁷. cDNA fragments for the two versions of the protease were separately cloned into the pcDNA 1.1 expression vector from Invitrogen (Carlsbad, Calif.). DNA corresponding to NS3/NS4A was cloned through a blunt end ligation and DNA corresponding to NS4A-NS3 was cloned into BamHI and EcoRI sites specifically. DNA fragments containing coding sequence of caspase 3, caspase 8 and BID were synthesized by PCR from Hela cDNA library (Invitrogen) with oligonucleotide primers corresponding to the 5′ end of Caspase 3. Modifications were introduced into the individual molecule using the QuickChange™ site-directed mutagenesis kit from Stratagene as previously described 48 Modified caspase and BID plasmids were isolated and the insertions of NS3 protease cleavage sites were confirmed by sequencing. CHOP cells at 50% confluency were cotransfected with different plasmids, using Geneporter (Gene Therapy Systems, San Diego, Calif.) and the relative fluorescence levels in the co-transfected cells were compared using FACS analysis.

Generation of Recombinant Retroviruses: NS3 protease (NS4A-NS3) was inserted between the BamHI and EcoRI of the retroviral expression vector, pBMN-IRES-GFP. Recombinant retrovirus was generated by introducing 10 μg of pBMN-NS3-IRES-GFP into 5×10⁶ cells of the ecotropic Phoenix packaging cell line using the calcium phosphate transfection method. Generation of high titer, helper-free retroviruses occurred following the transient transfection. Recombinant retrovirus was harvested at 48 hours post-transfection. H9C2 cells were infected at a multiplicity of infection (MOI) of 10 in 6-well microtiter plates (5×10⁵ cells/chamber). Cells were incubated for 24 hours before performing assays with modified BID.

Generation of Recombinant Adenovirus: Modified caspase 3 and BID were subcloned into PmeI site of pShuttle vector (Clontech, Palo Alto, Calif.) through blunt end ligation. The gene of interested was subsequently inserted into adenovirus genome using procedures described in the AdenoX expression Kit (Clontech, Palo Alto, Calif.). HEK-293 cells was transfected with 6 mg of recombinant adenovirus genome using GenePorter (Gene Therapy Systems, San Diego, Calif.), and recombinant adenovirus was isolated and amplified using procedures described by Clontech. Cells of interest were infected at a multiplicity of infection of 10 for at least 24 hours before performing assays.

Caspase Assays: 6-Well microtiter plates containing 1×10⁶H9C2 cells per chamber were grown to 50% confluency and infected with recombinant retrovirus expressing NS3 protease or EGFP. 24 hours after the initial retrovirus infection, cells were subsequently infected with control or recombinant adenovirus expressing modified caspase 3 or BID. After another 24 hours, cells were resuspended by dissociation solution (Sigma, St. Louis, Mo.) and collected by centrifugation at 400×g for 10 min. Pellets were resuspended in 100 μl of cold cell lysis buffer provided in ApoAlert caspase fluorescent assay kits (Clontech, Palo Alto, Calif.). Cell lysates were centrifuged at 18,000×g for 3 min at 4° C., and 50 μl of the supernatants were transferred to 96-well microtiter plates for detection of caspase 3 activities. The remaining portions of the supernatants were assayed for protein concentration. Caspase 3 activities were measured using fluorescent peptide substrates (DEVD-AFC) with a Wallac fluorimeter according to the ApoAlert kit instructions.

Apoptosis Analysis by Fluorescence Cytometry (FACS). H9C2 cells were infected and processed as described above in Caspase 3 Assay. Collected cells were centrifuged and washed twice with cold PBS and resuspended in 100 μl of binding buffer (PharMingen, La Jolla, Calif.). Subsequently, 5 μl of Annexin V-PE and 5 μl of 7-AAD (PharMingen) were added to the cell suspension and mixed gently. The cells were stained at room temperature in the dark for 15 min and analyzed with Becton Dickinson fluorescence cytometer using CellQuest software.

Western Immunoblot Analysis: H9C2 cells infected with both recombinant retrovirus expressing NS3 protease and recombinant adenovirus expressing modified BID was lysed with RIPA buffer followed by immunoprecipitation with Anti-FLAG M2-agarose affinity gel (Sigma, St. Louis, Mo.). SDS polyacrylamide gel electrophoresis and Western immunoblot analysis were performed. Anti-human BID monoclonal antibodies (1:1000 dilution) were incubated with nitrocellulose paper, and binding of monoclonal antibody was detected by ECL chemiluminescence (Amersham, Chicago, Ill.).

HCV genome Transfection and HCV Replicon cells: The coding region (nt. 339-nt. 9401) of HCV genome genotype 1a (obtained from Dr. C. Rice, Rockefeller University, New York, N.Y.) was subcloned into pcDNA1.1 expression vector through blunt end ligation. Huh7 cells were co-transfected with 5 μg of HCV genome and 1 μg of EGFP plasmids using GenePorter. 24 hours after transfection, the same cells were infected with either control adenovirus or recombinant adenovirus expressing modified BID at MOI of 10. Morphological changes of the infected Huh7 cells were evaluated under fluorescence microscope 36 hours after the adenovirus infection. 5×10⁵ each of the HCV replicon cells and their parental cells, BR-Huh7, were infected with either control adenovirus or recombinant adenovirus expressing modified BID at the MOI of 10. After 24 hours, morphological changes of the HCV replicon cells were observed under the phase-contrast microscope. In addition, HCV-genome transfected Huh7 cells and HCV replicon cells were lysed with SDS sample buffer, and Western blot analysis was performed with monoclonal antibody against HCV core protein (1:10000 dilution) and with monoclonal antibody against HCV NS3 protease (1:25). Binding of the monoclonal antibody was detected by ECL chemiluminescence (Amersham).

Chimeric Sindbis Virus Infection and Plaque Assay: Huh7 cells (5×10⁵) were infected with either control adenovirus or recombinant adenovirus expressing modified BID at MOI of 10. After 24 hours, same cells were infected with either wild-type Sindbis virus or chimeric Sindbis virus expressing HCV NS3 protease at MOI of 0.1. Infected cells were incubated at 30° C., and cell morphology was observed under phase-contrast microscope 72 hours post Sindbis virus infection. Supernatant of infected cells were collected at 72 hours post Sindbis virus infection and virus titers were determined by plaque assay as described by Filocamo et al. Plaques were stained with neutral red and counted 2 days after infecting 1×10⁵ BHK-21 cells in a 6 wells plates.

### Example 1

### Modification of Components of the FAS Apoptotic Pathway to Target the NS3/NS4A Protease of HCV

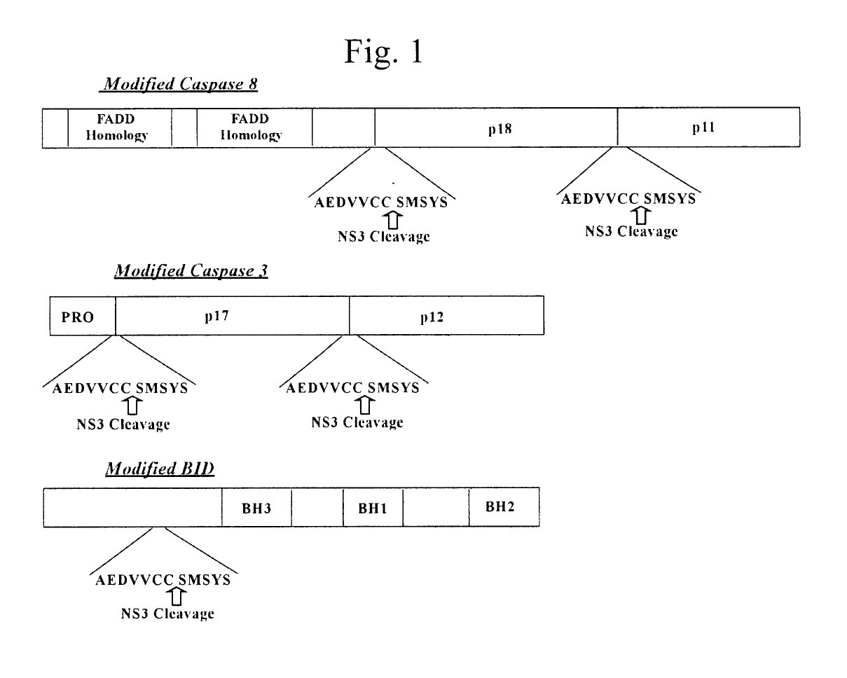

We modified the endogenous cleavage sites of procaspase 3 and BID by replacing them with an NS3/NS4A recognition site (FIG. 1A). The NS5A/NS5B cleavage sequence (AEDVVCCSMSYS; SEQ ID NO:20) was introduced to replace amino acids at positions positions 25 and 172 of procaspase 3, and position 62 of BID molecules using the QuickChange mutagenesis kit from Stratagene (FIG. 1) using a pro-caspase3 and pro-BID coding sequences (SEQ ID NO:28 and SEQ ID NO:26, respectively) as substrates for mutagenesis. In addition, the BID molecules have amino acid substitution at position 60 and 75 from aspartic acid (D) to glutamic acid (E) in order to remove the endogenous sites subject to cleavage by caspase 8 and granzyme B (Li et al., Cell 94, 491-501 (1998)). In addition a BID molecule that could not be cleaved was prepared as a negative control by mutating amino acids 68C69S to 68F69F and used this mutant to confirm that cleavage was required for apoptotic activity. All the constructs were initially cloned into pcDNA1.1 vector that expresses genes under the control of the CMV promoter and contains an origin of replication for cell lines with the T antigen.

### Example 2

### Transient Expression of Modified Caspase and BID Molecules in the Presence of NS3 Protease Results in Apoptosis

In order to determine whether modified apoptosis effectors can induce cell death in the presence of NS3 protease (i.e. the NS3/NS4A protease), CHOP cells were co-transfected with different modified caspase 3 or BID constructs along with vectors expressing NS3/NS4A protease and EGFP. Using FACS (fluorescence activated cells scanning) analysis, we were able to evaluate the amount of cell death by monitoring different levels of fluorescence in the cotransfected cells. Living cells contained EGFP and were fluorescent while dead cells released EGFP into the media resulting in a loss of fluorescence. Expression vectors containing modified procaspase 3, and BID were tested in this system in the presence and absence of NS3/NS4A protease. CHOP cells transfected with modified procaspase 3 alone appeared to have minimal cytotoxity in comparison to the native procaspase 3 by itself. However, co-transfection of a vector containing the coding sequence for procaspase 3 with a modified cleavage site at amino acid 172, and a plasmid expressing NS3/protease appeared to be cytotoxic. Cotransfections of procaspase 3 coding regions containing modified cleavage sites at amino acid 25 or both positions 25 and 172, together with the NS3 protease gene were less toxic in these assays. Thus, only introduction of NS5A/5B cleavage site at amino acid 172 of the procaspase 3 was found necessary to activate NS3 protease-dependent processing of procaspase 3.

The second apoptotic molecule we chose to modify was BID. This molecule is normally cleaved and activated by caspase 8 to stimulate the release of cytochrome c from the mitochondrion. CHOP cells transfected with the coding sequence of modified BID alone exhibited no cytotoxic effects. When CHOP cells were co-transfected with the coding regions of both modified BID and NS3 protease, cells were observed to die rapidly. The 68F69F mutant of BID that could not be proteolytically cleaved, could not initiate cell death in the presence or absence of NS3 protease. Based upon the preceding experiments, we generated recombinant adenoviruses expressing procaspase 3 and BID molecules with modified cleavage sites at positions 172 and 62 respectively.

Transient assays using CHOP cells transfected with coding regions for NS3/NS4A protease, EGFP, and modified procaspase or BID molecules were used to determine whether different constructs can induce cell death in the presence of viral protease. After 24 hrs, cell death was scored through FACS analysis of living EGFP positive cells. (−) indicates no cell death, (+) indicates 5%-15% cell death, and (+++) indicates >30% cell death. In similar experiments it was found that the cleavage site at Asp position 98 of BID should be altered to prevent proteolytic cleavage at that site in order to prevent toxicity is cells that do not express NS3 protease. These data are shown in Table 2.

TABLE 2
Analysis of Cell Death Using Transient Expression Assays

| | (−) NS3 | (+) NS3 |
|---|---|---|
| Plasmid Transfected | Protease | Protease |
| Wild Type Caspase 8 | (+++) | (+++) |
| Mod.Caspase 8/207(NS5A/B) | (+++) | (+++) |
| Mod.Caspase 8/371(NS5A/B) | (+++) | (+++) |
| Mod.Caspase 8/207/371(NS5A/B) | (+++) | (+++) |
| Mod.Δ178Caspase 8/207(NS5A/B) | (+) | (+) |
| Mod.Δ178Caspase 8/371(NS5A/B) | (+) | (+) |
| Mod.Δ178Caspase 8/207&371(NS5A/B) | (+) | (+) |
| Wild Type Caspase 3 | (+) | (+) |
| Mod.Caspase 3/25(NS5A/B) | (−) | (+) |
| Mod.Caspase 3/172(NS5A/B) | (−) | (+++) |
| Mod.Caspase 3/25&172(NS5A/B) | (−) | (+) |
| Wild Type BID | (+++) | (+++) |
| Mod.BID/60(NS5A/B) | (−) | (+++) |

### Example 3

### Recombinant Adenoviruses Expressing Modified Procaspase 3 and BID Molecules Target a Rat Cell Line that Constitutively Synthesizes NS3/NS4A Protease

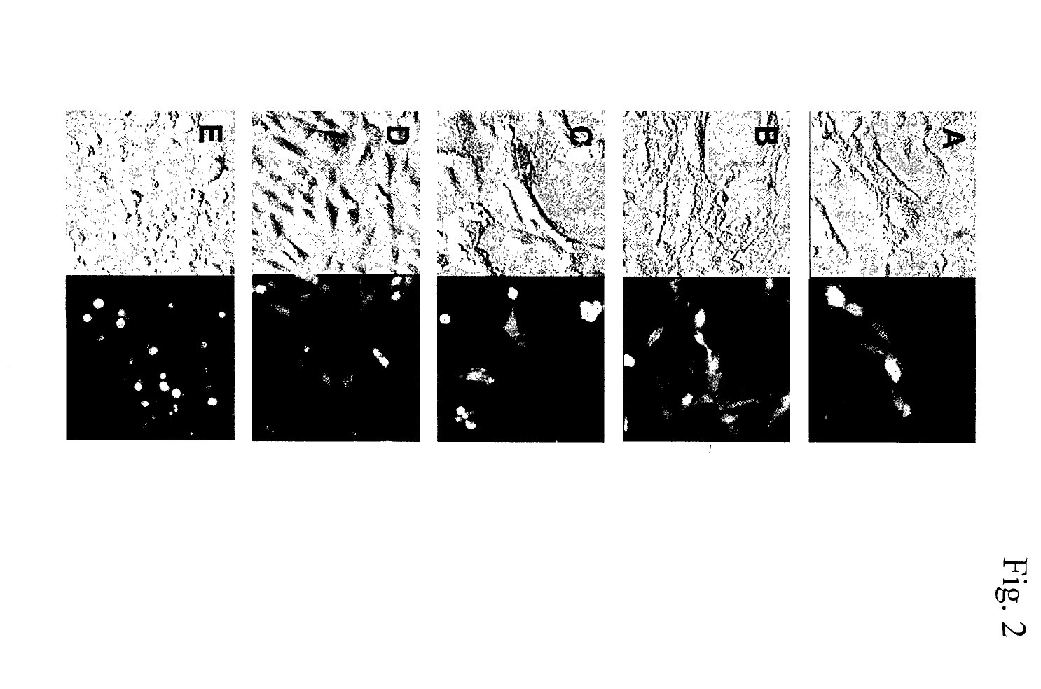

Recombinant adenoviruses that expressed either modified procaspase 3 or modified BID were generated with the Adeno-X system from Clontech. In addition, we also constructed a recombinant retrovirus that expressed HCV NS3 protease (NS4A-NS3) under control of the LTR promotor, and EGFP from the IRES internal translation signal. Rat muscle fibroblast cell (H9C2) were infected with recombinant retroviruses that expressed either NS3 protease and EGFP, or EGFP alone. Approximately 24 hours following retrovirus infection, the same cells were infected with either control or recombinant adenovirus that expressed either modified procaspase 3 or modified BID. The H9C2 cells were chosen for these experiments since they can be infected efficiently by both ecotropic strains of retrovirus as well as human adenovirus. After another 24 hours, infected H9C2 cells were visualized under the fluorescence microscope, photographed, and morphological changes were noted (FIG. 2). H9C2 cells expressing either NS3 protease alone, modified caspase3 alone, or modified BID alone, appeared to have normal morphology when visualized under either polarized light or UV fluorescence microscopy (FIGS. 2A, 2B, 2D). However, cells that expressed both NS3 protease and modified procaspase 3 or modified BID, exhibited membrane blebbing and condensation of the nuclei, visual indicators of apoptosis (FIGS. 2C, 2E). Although both modified procaspase 3 and modified BID molecules induced cell death in the presence of NS3 protease, modified BID appeared to be the potent activator of the apoptotic pathway.

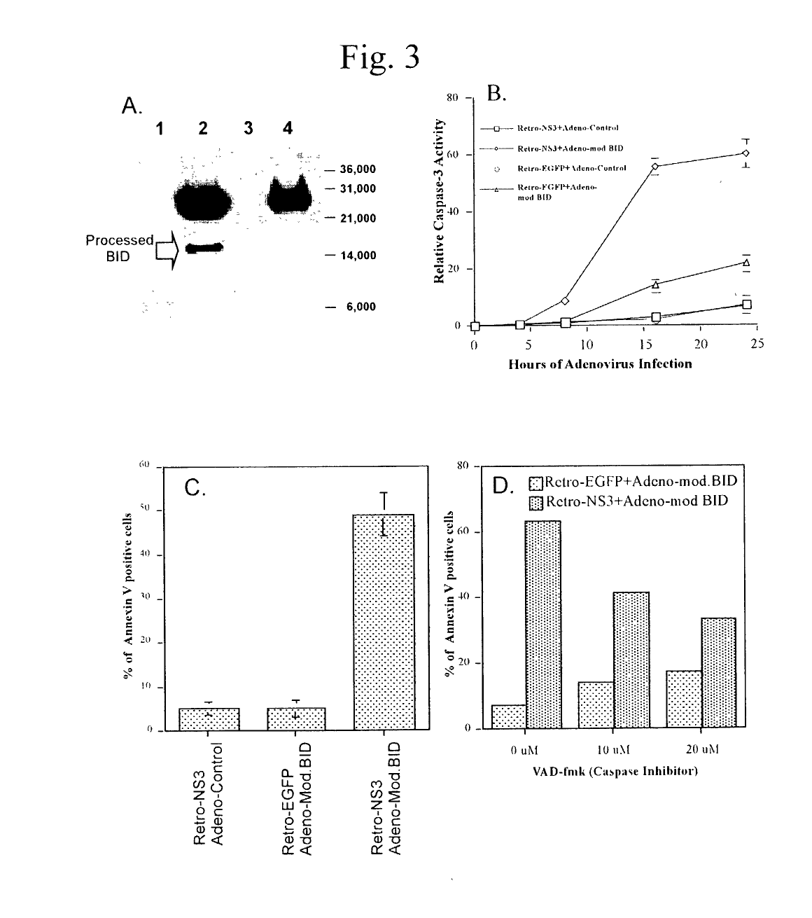

In order to verify that modified BID molecules were actually processed and activated by the NS3 protease. H9C2 cells were infected first with recombinant retrovirus that expressed either NS3 protease and EGFP or EGFP alone. After 24 hours, the same cells were subsequently infected with control adenovirus or recombinant adenovirus that expressed modified BID. In these experiments the modified BID molecule was engineered to contain a FLAG epitope tag at its carboxyl terminus. Following a further 24 hours, infected cells were lysed and immunoprecipitation was performed with FLAG antibodies coupled to sepharose beads. Protein products were resolved by PAGE and detected by immunoblot analysis using a polyclonal BID antibody (FIG. 3A). Cells that expressed modified BID alone generated a single 24 kDa fragment characteristic of the uncleaved BID molecule (lane 4). On the other hand, cells that expressed both modified BID and NS3 protease contained the 24 kDa uncleaved fragment as well as a 16 kDa cleaved fragment, indicating that modified BID molecules were successfully processed by the NS3 protease.

We also confirmed that the apoptotic pathway was activated by NS3-processed BID. Cells containing or lacking NS3 protease were lysed at different times following adenovirus infection and their caspase 3 activities were measured using the Fluorescence Caspase 3 Activity Assay Kit from Clonetech. H9C2 cells expressing both NS3 protease and modified BID exhibited increasing caspase 3 activity beginning around 8 hours post-infection (FIG. 3B). However, cells expressing NS3 alone or modified BID alone appeared to have low levels of caspase 3 activity. H9C2 cells infected with “empty” retrovirus and adenovirus controls contained negligible caspase 3 activity. The amount of apoptosis in H9C2 cells expressing NS3 proteas, modified BID or both, was quantitated with AnnexinV staining at 24 hours following infection with recombinant adenovirus. AnnexinV is a marker for apoptosis that labels phosphatidyl serine on the exterior of cells undergoing apoptosis and staining is quantitated with AnnexinV antibodies using FACS. When both recombinant NS3 and modified BID were expressed, 50% more cells were AnnexinV positive than those infected with control virus, while cells expressing either the NS3 alone or modified BID alone were only 2-5% above the control background (FIG. 3C). Finally, apoptosis generated by the co-expression of NS3 protease and modified BID could be reversed with a caspase inhibitor, VAD-fmk. H9C2 cells infected with NS3 or control retroviruses were subsequently infected with recombinant adenovirus expressing modified BID and were treated with different concentrations of VAD-fmk. The number of apoptotic cells was determined by AnnexinV staining after 24 hours of recombinant adenovirus infection. Again, cells expressing modified BID alone in the absence of VAD-fmk were only 5% AnnexinV positive (FIG. 3D). H9C2 cells that expressed both NS3 protease and modified BID, were 60% AnnexinV positive. With the addition of 10 μM and 20 μM VAD-fmk inhibitor, the number of AnnexinV positive cells dropped to 42% and 34%, respectively (FIG. 3D). Since caspase 3 activity and annexinV staining were elevated, these results confirmed our hypothesis that apoptosis is induced in target cells containing the HCV protease (NS3) and modified BID.

### Example 4

### Hepatocytes Expressing the HCV Genome and Modified BID Undergo Apoptosis

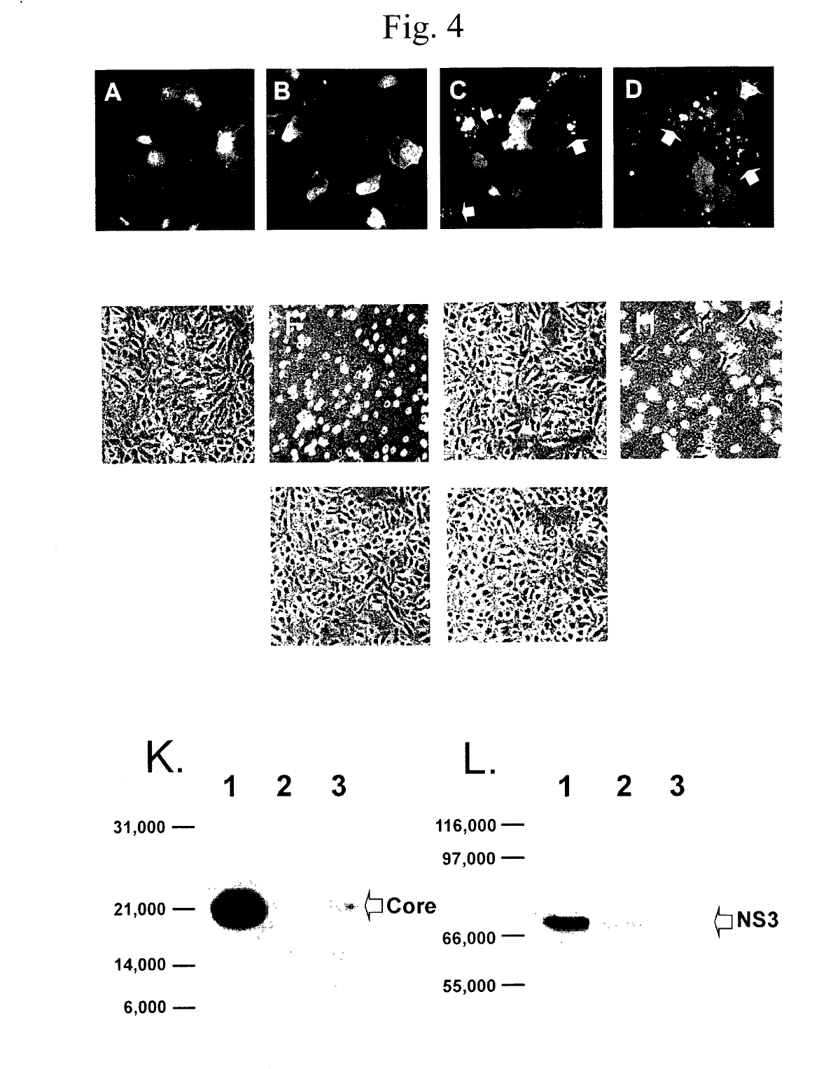

We decided to test whether the above strategy could selectively induce apoptosis in human hepatocytes that contained the HCV viral genome. Since there is no culture system for hepatitis C virus, we co-transfected human hepatocytes (Huh7) with an expression plasmid that expressed the entire HCV genome and another reporter vector that expressed EGFP. The presence of processed HCV viral proteins in the transfected cells was confirmed by immunoblot analysis using monoclonal antibodies against the HCV core and NS3 proteins (FIG. 4K, lane 3; FIG. 4L, lane 2). At 48 hours following transfection, the Huh7 cells were infected with either control adenovirus or recombinant adenovirus that expressed modified BID. Cells were observed by UV fluorescence microscopy after 36 hours of adenovirus infection. Huh7 cells transfected with a cDNA copy of the HCV genome or infected with recombinant adenovirus expressing modified BID alone, displayed no morphological change (FIGS. 4A, 4B). However, Huh7 cells that expressed modified BID and either the HCV genome or NS3 protease, exhibited membrane blebbing, nuclear condensation, and cellular disintegration, clear indications of cellular apoptosis (FIGS. 4C, 4D). Even relatively small amounts of NS3 protease were capable of activating modified BID to induce apoptosis.

### Example 5

### Hepatocytes Containing HCV Replicons Undergo Apoptosis in the Presence of Modified BID

Two groups have reported the development of HCV replicon systems in the Huh7 hepatocyte cell line (Lohmann, et al. Science 285, 110-113. (1999); Blight et al. Science 290, 1972-1974 (2000)). These systems consist of the 5′ UTR of HCV, a neomycin gene for selection, the nonstructural genes of HCV under control of an IRES from encephalomyocarditis virus (EMC), and the 3′ UTR of HCV. Nonstructural proteins, including the NS3 protease, are translated and cleaved in this system. Two different HCV replicon cell lines (HBI-10A and HBIII-27), as well as parental Huh7 cells, were infected with either recombinant adenovirus expressing modified BID or control adenovirus. At 24 hours infection, cells were observed under the phase contrast microscope (FIGS. 4E-4J). Both replicon cell lines and Huh7 cells exhibited no morphological changes following infection with control adenovirus (FIGS. 4E, 4G, 4I). Similarly, Huh7 cells infected with adenovirus expressing modified BID also yielded no visible changes, indicating that modified BID by itself had little toxicity in these cells (FIG. 4J). On the other hand, both HBI-10A and HBIII-27 replicon cell lines exhibited severe cytotoxicity following infection with adenovirus expressing modified BID (FIGS. 4F, 4H). Cell death was quantitated using trypan blue staining. The parental Huh7 cells exhibited less than 5% cell death following infection with the modified BID adenovirus, but the infection of the replicon cell lines, HBI-10A and HBIII-27, with the same recombinant virus killed 87% and 80% of the cells after 24 hours (data not shown). These results demonstrated that the above described system can selectively destroy human hepatocytes containing the nonstructural proteins of HCV derived from a replicon system.

### Example 6

### Modified BID Prevents Infection of Huh7 Cells with a Chimeric Sindbis Virus that Expresses the NS3 Protease of HCV

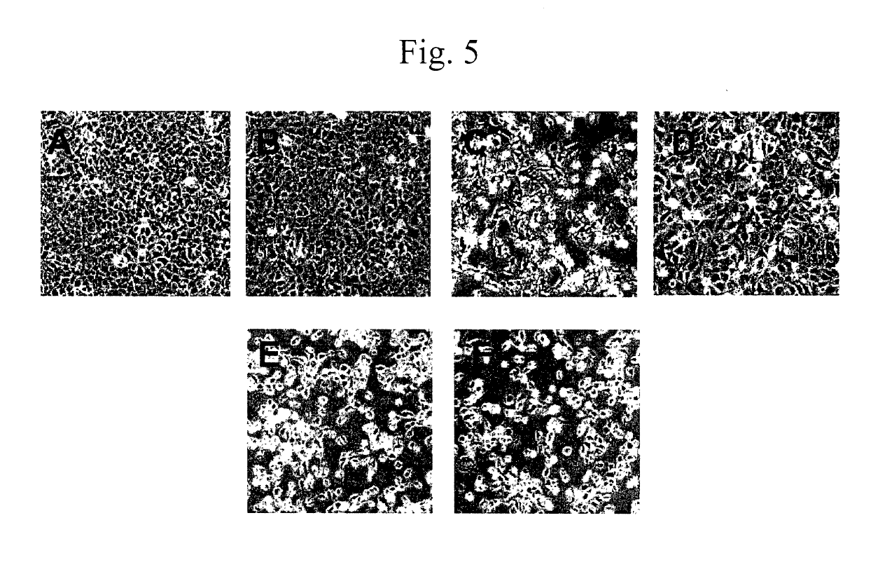

Although our gene therapeutic approach with modified BID can successfully activate apoptosis in cells expressing NS3 protease, one of the key concerns for using this system is that increased amounts of virus may be released during apoptosis that could subsequently infect other cells. On the other hand, pretreatment of cells with modified BID prior to HCV infection may be prophylactic, and decrease the viral load through induction of apoptosis before viral assembly. In order to test this theory, we obtained a model for HCV consisting of a chimeric Sindbis virus (MutA) that synthesized a polyprotein composed of HCV-NS3 fused to the amino terminus of the precursor polypeptide for the structural proteins of Sindbis virus (NS3-C-PE2-6K-E1). The NS3-C and C-PE2 junctions were engineered to contain the cleavage site for NS3 protease (EDVVCC/SMSY) and processing of this site by the HCV protease was required for assembly of Sindbis virus. Thus, chimeric Sindbis virus (MutA) required NS3 activity in order to replicate in cell culture. In our experiments, Huh7 cells were initially infected with either control adenovirus or recombinant adenovirus expressing modified BID. After 24 hours, the same cells were subsequently challenged with either wild type Sindbis virus or chimeric Sindbis virus (MutA). Lysis of infected cells by processed Sindbis virus was assessed at 72 hours infection using a phase contrast light microscope. Huh7 cells infected with either control adenovirus or recombinant adenovirus expressing modified BID alone appeared normal under the microscope (FIGS. 5A, 5B). On the other hand, cells infected with wild type Sindbis virus appeared to be sparse, fibrous, and round in appearance, irrespective of the presence of modified BID (FIGS. 5E, 5F). Huh7 cells infected with control adenovirus and chimeric Sindbis (MutA) exhibited the same cytopathic effects as cells infected with wild type Sindbis virus (FIG. 5C). However, cells pre-incubated with adenovirus expressing modified BID and subsequently challenged with chimeric Sindbis (MutA) appeared to be normal (FIG. 5D). This result indicated that pre-treatment of cells with modified BID could subsequently protect cells from chimeric Sindbis virus infection. The prophylactic effect of modified BID against challenge by chimeric Sindbis virus was quantitated by plaque assay (FIG. 5G). These results suggest that modified BID induces apoptosis following activation by HCV NS3 protease, and prevents the subsequent replication and assembly of Sindbis virus. Thus, the modified BID therapeutic system may have prophylactic potential in reducing the overall viral load of HCV in the liver, prior to, or during early and limited stages of infection.

In addition, Huh7 cells were infected with either control adenovirus or recombinant adenovirus expressing BID at a multiplicity of infection (MOI) of 10 pfu/cell. 24 hours later, the same cells were challenged with either wild type Sindbis virus (SBV) or NS3 chimeric Sindbis virus (SBV MutA) at a MOI of 0.1 pfu/cell. Supernatants were collected at 72 hrs following infection with Sindbis virus. Titers were determined by plaque assay using BHK-21 cells after staining cells 2 days later with neutral red. An average titer of Sindbis virus (pfu, plaque forming units) was determined from four independent assays and displayed in Table 3.

TABLE 3
Plaque Assays Using NS3 Chimeric
Sindbis Virus In Presence of Modified BID

| Type of Adenovirus/Sindbis Added | Sindbis Virus Produced (pfu) |
|---|---|
| Adeno-Control + SBV(wild type) | 6.5 × 10⁶ |
| Adeno-Mod.BID + SBV(wild type) | 5.5 × 10⁶ |
| Adeno-Control + SBV(Mut A) | 3.0 × 10⁵ |
| Adeno-Mod.BID + SBV(MutA) | 7.8 × 10³ |

### Example 6

### Reduction of HCV Viral Load in HCV Infected Mice

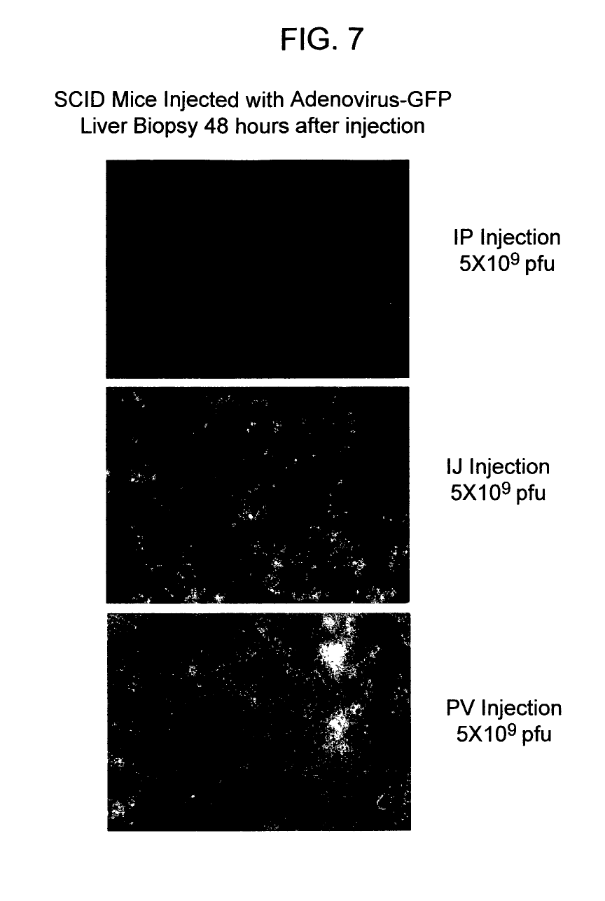

Mice having a chimeric liver with human hepatocytes were inoculated HCV, as described in PCT publication WO0167854. In order to determine the best route of administration of a mod-BID construct (encoding SEQ ID NO:2), 5×10⁹ pfu of an adenovirus encoding GFP was injected intraperitoneally (IP), intrajugularly (IJ) and into the portal veins (PV) of test mice. The livers of these mice were examined under fluorescence (FIG. 7) and it was determined that intrajugular and portal vein injections were a suitable administration route.

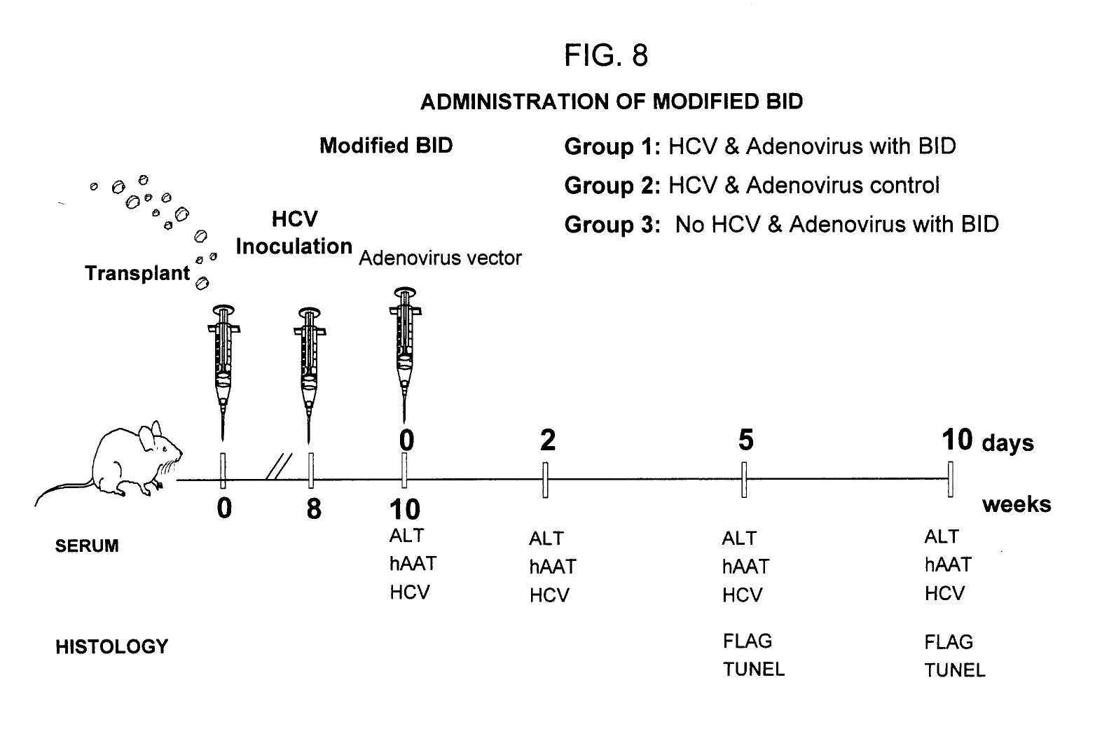

These HCV infected mice are injected IJ or IP with a modified BID containing a HCV protease cleavage sequence in an adenovirus vector at the designated times, shown in FIG. 8. Liver cells were transplanted at week 0, HCV was inoculated at week 8 and at week 10, injections of approximately 5×10⁹ pfu adenoviral vector encoding modified BID were performed (day 0). Mice were generally divided into three groups: Group I that was administered HCV and a modified BID-encoding vector, Group 2 and Group 3 were controls. Approximately 2 to 5 mice were in each group. Liver damage due to apoptosis or viral infection was measured by measuring ALT (alanine amino transferase) activity and human alpha anti-trypsin (HAAT) activity in serum; serum human alpha 1 anti-trypsin also provided a measure of residual functional human hepatocytes after treatment. Serum viral loads (HCV) were measured by RT-PCR with the Roche Amplicor System as blinded samples at a third party lab (the Provincial Laboratory of Public Health of Alberta). Virus titers were expressed on a log scale as units of genomic RNA. At days 5 and 10, the FLAG epitope, found in one modified BID polypeptide, was measured to show that BID is being expressed. TUNEL assays were used to measure apoptosis of liver cells as confirmation of mechanism.

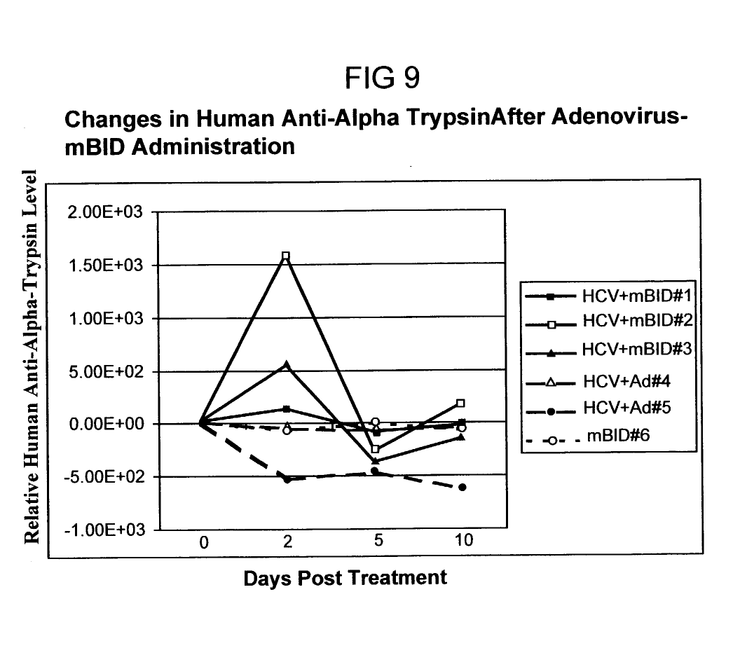

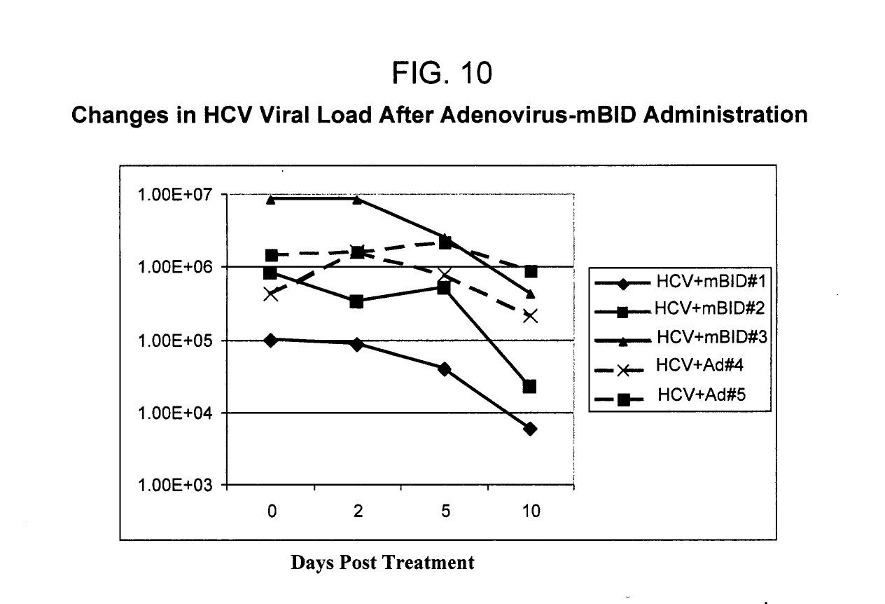

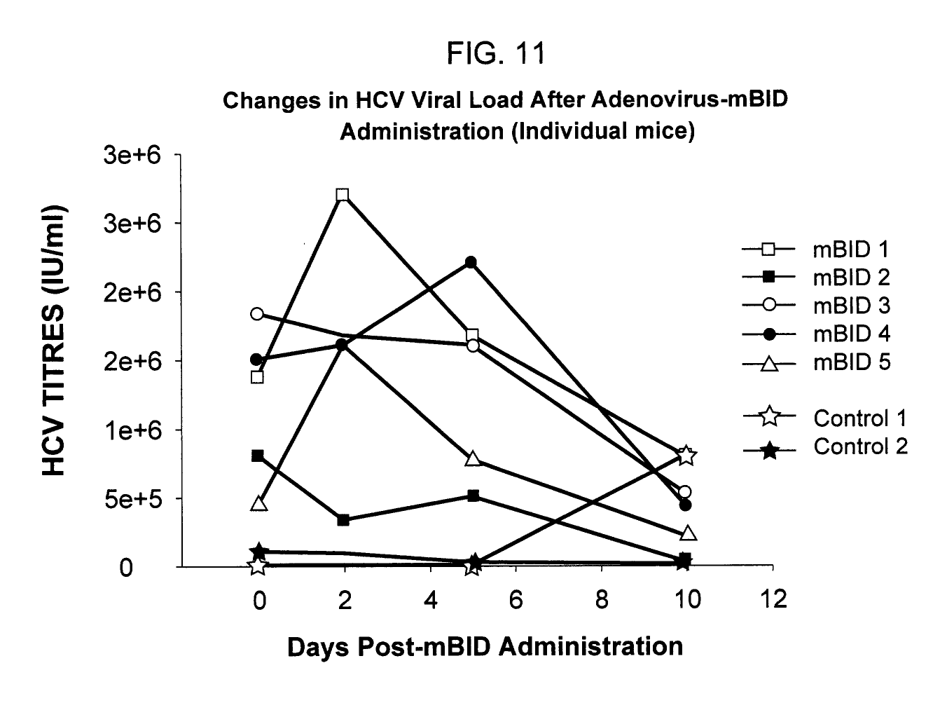

Mice infected with HCV contained different degrees of HCV infection and consequently had different levels of genomic RNA. Mice treated with modified BID adenovirus exhibited an increase in ALT levels due to apoptosis of HCV-infected liver cells by modified bid (FIG. 9) as compared to control mice. Mice treated with modified BID exhibited an decrease in serum HCV of up to 1.6 logs (i.e. greater than 95%) (FIG. 10). Experiments with more mice (5) confirmed these results (results shown on FIG. 11). As such, the viral load of mice infected by HCV was reduced by the subject nucleic acids.

### Example 7

### Reduction of HCV Viral Load in HCV Infected Mice II

In a separate experiment, four mice were infected with HCV and treated with a modified BID-encoding vector (mBID), as described in Example 6 above. Seven other mice acted as controls. ALT activity, HAAT activity and viral titer was measured for all mice.

Viral titers for mice infected with HCV and treated with mBID are presented in Table 4, below:

TABLE 4

| | Initial viral | Final |
|---|---|---|
| Mouse | titer per ml | viral titer |
| 952 | 8.07 × 10³ | <600* |
| 998 | 3.50 × 10⁴ | <600* |
| A17 | 4.94 × 10⁷ | 1.5 × 10⁵ |
| A77 | 3.80 × 10E5 | 5.3 × 10⁵ |

*<600 is undetectable

Mice A17 and A77 died after serum was isolated, and controls did not show significant reductions of viral titer.

As such, in two mice or four, viral load was reduced to an undetectable level from approximately 1×10⁴ pfu per ml.

In vivo data comparing mod-BID with and without the D98E modification were compared, and shown in Table 5:

TABLE 5

| | | | | Viral titre |
|---|---|---|---|---|
| M-BID | mouse | Day 0 | day 1 | reduction |
| D98E | 141 | 1.00 × 10⁵ | 5.95 × 10³ | (16.8X) |
| D98E | 142 | 8.00 × 10⁵ | 2.25 × 10⁴ | (35.6X) |
| D98E | 144 | 1.83 × 10⁶ | 5.2 × 10⁵ | (3.52X) |
| D98E | 152 | 1.37 × 10⁶ | 7.9 × 10⁵ | (1.73X) |
| D98E | 952 | 8.07 × 10³ | <600* | (>13.5X) |
| D98E | 998 | 3.50 × 10⁴ | <600* | (>58.3X) |
| D98E | a17 | 4.94 × 10⁷ | 1.7 × 10⁵ | (291X) |
| Control | 150 | 4.3 × 10⁵ | 2.10 × 10⁵ | (2.0X) |
| Control | 151 | 1.5 × 10⁶ | 4.3 × 10⁵ | (3.49X) |
| Control | 158 | 0.6 × 10³ | 0.69 × 10³ | (0.9X) |
| Control | 159 | 1.19 × 10³ | 0.6 × 10³ | (2.0X) |
| M-BID | 140 | 1.56 × 10⁶ | 8.2 × 10⁵ | (1.92X) |
| MBID | 146 | 8.5 × 10⁶ | 7.5 × 10⁵ | (11.3X) |
| MBID | 149 | 7.70 × 10⁵ | 4.3 × 10⁵ | (1.79X) |

*<600 is undetectable

Mod-BID containing the D98E mutation was more effective than mod-BID not containing the D98E mutation by a by Wilcoxon rank sum method gives (P<0.05).

### Example 8

### Reduction of Viability of HIV Infected Cells

To show the modified pro-polypeptides of the invention can specifically kill HIV-infected cells, adenoviral vectors encoding an HIV protease site modified BID (SEQ ID NOS: 3 and 27) are tested.

About 5×10⁶ Jurkat T-cells are infected with HIV (strain NLHX; about 1×10⁵ to 1×10⁶ infectious virus per ml). The cells are propagated in RPMI media. Approximately 4 to 7 days after the infection, the media was removed from the plates and, following the same protocol as above, the cells are inoculated with an adenoviral vector encoding a modified BID-polypeptide with an HIV cleavage sequence (shown in FIG. 6C and in SEQ ID NOS: 3 and 27). It is expected that approximately 100% of the HIV infected cells are killed using this method. As a control, the vectors are also used to infect cells not infected with HIV. In this control, less than 1% of the cells will be killed.

### Example 9

### Reduction of HIV Viral Load in HIV Infected Mice

Mice are prepared and infected with HIV according to the methods provided by U.S. Pat. No. 5,612,018. These HIV infected mice are injected IJ or IP with an adenoviral vector encoding a modified BID containing a HIV protease cleavage sequence as shown in FIG. 6C at designated times. HIV viral load and CD4 count are measured. HIV titer is reduced and CD4 count is increased in the plasma of these mice.

### Example 10

### Diagnosis of HIV and HCV

Vectors of the invention are used in a diagnosis method for HIV or HCV. In this method, a test cell that may be infected with an intracellular pathogen (e.g. HIV or HCV) is contacted with a subject nucleic acid encoding a modified pro-polypeptide with a suitable protease cleavage site for the pathogen. If a cell contacted with a subject nucleic exhibits a decrease in viability (e.g. dies or performs apoptosis), is may be identified as a cell infected with a particular pathogen (e.g. HIV or HCV). In many embodiments, a plasmid vector encoding the subject pro-polypeptide and a method for detecting cellular apoptosis, such as TUNEL, are used.

It is evident from the above results and discussion that the subject invention provides an important new means for treating diseases caused by intracellular pathogens. In particular, the subject invention provides a system for treating a subject with a nucleic acid encoding a modified pro-polypeptide engineered with a pathogen protease dependent cytotoxic molecule. As such, the subject methods and systems find use in a variety of different applications, including research, medical, therapeutic and other applications. Accordingly, the present invention represents a significant contribution to the art.

What is claimed is:

1. A nucleic acid comprising a nucleotide sequence encoding a modified pro-polypeptide, the modified pro-polypeptide having a sequence-specific protease cleavage site operably inserted between a pro-domain of the pro-polypeptide and a mature polypeptide domain of the pro-polypeptide,
wherein the protease cleavage site is not native to said pro-polypeptide and wherein the mature polypeptide domain is cytotoxic upon cleavage of said pro-polypeptide at the protease site.

2. The nucleic acid according to claim 1, wherein said protease site is a site for a viral sequence-specific protease.

3. The nucleic acid according to claim 1, wherein a sequence-specific protease cleavage site that is native to said pro-polypeptide is modified to prevent cleavage by a cellular protease.

4. The nucleic acid according to claim 3, wherein said protease site is a site for a viral sequence specific protease chosen from cytomegalovirus, herpes simplex virus type-1, hepatitis virus type C (HCV), human immunodeficiency virus type 1, human immunodeficiency virus type 2 and Kaposi's syndrome associated herpes virus protease cleavage sites.

5. The nucleic acid according to claim 3, wherein said protease cleavage site is for a HCV-encoded protease.

6. The nucleic acid according to claim 1, wherein said protease site is chosen from SEQ ID NOS:8-21 and SEQ ID NOS:23-25.

7. The nucleic acid according to claim 1, wherein said cytotoxic activity is an induction of apoptosis.

8. The nucleic acid according to claim 1, wherein said pro-polypeptide is a caspase pro-polypeptide or a BH3 interacting domain death agonist (BID) pro-polypeptide.

9. A nucleic acid comprising a nucleotide sequence encoding a modified BH3 interacting domain death agonist pro-polypeptide (pro-BID), the modified pro-BID having a sequence-specific protease cleavage site operably inserted between a pro-domain of the pro-BID and a mature BID domain of the pro-BID,
wherein the protease cleavage site is not native to said pro-BID and wherein the mature BID domain is cytotoxic upon cleavage of said pro-BID at the protease site.

10. The nucleic acid according to claim 9, wherein the modified pro-BID is modified at a granzyme B protease recognition site such that the modified granzyme B protease recognition site is not cleaved by granzyme B.

11. The nucleic acid according to claim 10, wherein the Asp amino acid residue at position 98 of the pro-BID is modified to any other naturally occurring amino acid residue.

12. The nucleic acid according to claim 11, wherein said other naturally occurring amino acid is a Glu amino acid residue.

13. The nucleic acid according to claim 11, wherein further Asp amino acid residues at positions 59 and 75 are modified to any other naturally occurring amino acids.

14. The nucleic acid according to claim 11, wherein said other naturally occurring amino acids are Glu amino acid residues.

15. The nucleic acid according to claim 9, wherein said sequence specific protease recognition site is a site for an HIV protease or a HCV protease.

16. The nucleic acid according to claim 15, wherein the modified pro-BID has the sequence chosen from SEQ ID NO:2, SEQ ID NO:3, and SEQ ID NO:27.

17. A pro-polypeptide encoded by the nucleic acid of claim 1.

18. An expression cassette comprising the nucleic acid of claim 1.

19. A vector comprising the expression cassette of claim 18.

20. The vector according to claim 19, wherein said vector is an adenoviral vector.

21. A host cell comprising the vector of claim 19.

22. A method of reducing the viability of a cell expressing a protease encoded by a pathogen, said method comprising:
introducing the vector according to claim 20 into said cell, wherein said vector encodes a pro-polypeptide comprising a recognition site for said protease; and
incubating said cell under conditions to allow for expression of said pro-polypeptide.

23. The method according to claim 22, wherein said cell is infected by a pathogen and said protease is a pathogen-encoded protease.

24. The method according to claim 23, wherein said pathogen is a viral pathogen.

25. The method according to claim 24, wherein said viral pathogen is a HCV pathogen and said protease is an HCV protease.

26. The method according to claim 24, wherein said viral pathogen is an HIV-1 pathogen and said protease is an HIV-1 protease.

27. The method according to claim 22, wherein said cell is a cell of a liver to be transplanted into a liver recipient.

28. A method for determining the effect of a modified propolypeptide on the viability of a cell expressing a pathogen-encoded protease, said method comprising:
introducing the vector according to claim 20 into said cell, wherein said vector encodes a pro-polypeptide comprising a recognition site for said protease;
incubating said cell under conditions to allow for expression of said pro-polypeptide; and,
determining the viability of said cell.

29. A method of reducing a pathogen load of a subject infected with a pathogen encoding a protease, said method comprising:
administering the vector according to claim 20 to said subject, wherein said vector encodes a pro-polypeptide comprising a recognition site for said protease.

30. The method according to claim 29, wherein said pathogen is a viral pathogen.

31. The method according to claim 30, wherein said viral pathogen is HCV and said protease is an HCV protease.

32. The method according to claim 30, wherein said viral pathogen is HIV-1 and said protease is an HIV-1 protease.

33. The method according to claim 29, wherein said administering is systemic.

34. The method according to claim 29, wherein said administering step is into a jugular vein or a portal vein.

35. The method according to claim 29, wherein said subject is a organ transplant recipient.

36. The method according to claim 35, wherein said organ is a liver.

37. A kit comprising:
the nucleic acid of claim 1; and
instructions for the use of said nucleic acid for the treatment of a disease associated with expression of a protease.
